(19) United States
(12) Patent Application Publication
(10) Pub. No.: US 2026/0191884 A1
(43) Pub. Date: Jul. 9, 2026

(54) SYSTEMS AND METHODS FOR IMPROVING THE SAFETY AND EFFICACY OF TREATMENT USING CANNABIS-DERIVED SUBSTANCES WITH A VULNERABLE POPULATION

(71) Applicant: HJK Medical Enterprises LLC, Palm Beach, FL (US)

(72) Inventor: Howard J. Kessler, Palm Beach, FL (US)

(21) Appl. No.: 19/128,295

(22) PCT Filed: Nov. 6, 2023
(86) PCT No.: PCT/US2023/078792

§ 371 Date: May 8, 2025

Related U.S. Application Data
(60) Provisional application No. 63/383,158, filed on Nov. 10, 2022.
(60) Provisional application No. 63/508,587, filed on Jun. 16, 2023.

(51) Int. Cl. A61K 31/00; G16H 20/10; G16H 50/30

(52) U.S. Cl. CPC A61K 31/658; G16H 20/10; G16H 50/30

(57) ABSTRACT

A treatment method may include administering a first cannabinoid-based treatment to a first subject; receiving feedback from the first subject after the first cannabinoid-based treatment has been administered; adjusting the first cannabinoid-based treatment to provide a second cannabinoid-based treatment in response to the feedback; and administering the second cannabinoid-based treatment to the first subject. Systems, devices, and non-transient machine-readable storage media also are disclosed.

### CROSS-REFERENCE TO RELATED APPLICATIONS

This application claims priority to U.S. Provisional Patent Application No. 63/508,587, filed Jun. 16, 2023, and to U.S. Provisional Patent Application No. 63/383,158, filed Nov. 10, 2022, the entireties of which are incorporated by reference herein.

### FIELD

The present disclosure generally relates to medical research. More specifically, the disclosed systems and methods relate to medical research performed on a vulnerable population to improve the safety and efficacy of treatment of individuals using cannabis-derived substances.

### BACKGROUND

Although cannabis has been used for medical purposes for thousands of years, the endocannabinoid system (“ECS”) was only recently identified and has been minimally studied. Endogenous cannabinoids, also referred to as endocannabinoids, are lipid-based neurotransmitters that are naturally occurring in the human body. Phytocannabinoids, also referred to as exogenous cannabinoids, are plant-derived cannabinoids that naturally occur in the cannabis plant. The two most well-known phytocannabinoids are tetrahydrocannabinol (“THC”) and cannabidiol (“CBD”). Phytocannabinoids may be used to support a body's ECS to maintain homeostasis. However, due to cannabis being a Schedule 1 drug, little research has been done.

### SUMMARY

In some embodiments, a system may include a non-transitory machine-readable storage medium and a processor in communication with the non-transitory machine-readable storage medium. The processor may be configured to receive first data concerning a first subject's experience with a first cannabinoid-based treatment, store the first data to be stored in the non-transitory machine-readable storage medium in a profile associated with the first subject, identify at least one change to be made to the first cannabinoid-based treatment, and cause at least one message identifying a second cannabinoid-based treatment to be generated in response to identifying the at least one change to be made to the first cannabinoid-based treatment.

In some embodiments, the processor may be configured to identify the second cannabinoid-based treatment in response to analyzing the first data.

In some embodiments, the second cannabinoid-based treatment may differ from the first cannabinoid-based treatment by an amount of at least one active ingredient.

In some embodiments, the second cannabinoid-based treatment may differ from the first cannabinoid-based treatment by a dosing regimen.

In some embodiments, the processor may be configured to identify the second cannabinoid-based treatment in response to analyzing second data concerning a second subject's experience with the first cannabinoid-based treatment.

In some embodiments, the second cannabinoid-based treatment may differ from the first cannabinoid-based treatment by an amount of at least one active ingredient.

In some embodiments, the second cannabinoid-based treatment may differ from the first cannabinoid-based treatment by a dosing regimen.

In some embodiments, the second data may be stored in the non-transitory machine-readable storage medium in a profile associated with the second subject.

In some embodiments, the first data may be received from a computing device associated with the first subject.

In some embodiments, the at least one message may be transmitted to the computing device associated with the first subject.

In some embodiments, the first data may be received from a first sensor associated with the first subject.

In some embodiments, the at least one message may be transmitted to at least one of a medical professional and a member of an advisory board.

In some embodiments, the first subject may be at least 65 years old.

In some embodiments, the processor may be configured to receive real-world data concerning a plurality of cannabinoid-based treatments from a plurality of subjects.

In some embodiments, the processor may be configured to create a profile for each subject of the plurality of subjects in the non-transitory machine-readable storage medium.

In some embodiments, each subject of the plurality of subjects may be at least 65 years of age.

In some embodiments, a system may include a first computing device. The first computing device may include an input device, a display, a communication interface, and a first processor in communication with the input device, the display, and the communication interface. The first processor may be configured to receive, from the input device, first data concerning a first subject's experience with a first cannabinoid-based treatment; cause the first data to be transmitted, using the communication interface, to a second computing device; receive, using the communication interface, a message identifying a second cannabinoid-based treatment; and cause a notification identifying the second cannabinoid-based treatment to be displayed on the display.

In some embodiments, the second cannabinoid-based treatment may differ from the first cannabinoid-based treatment by an amount of at least one active ingredient.

In some embodiments, the second cannabinoid-based treatment may differ from the first cannabinoid-based treatment by a dosing regimen.

In some embodiments, the first computing device may be configured to receive health data from a sensor associated with the first subject.

In some embodiments, the health data may include at least one of a pulse rate, a blood pressure, and a blood-oxygen level.

In some embodiments, the health data may include data about the first subject's sleep.

In some embodiments, a treatment method may include administering a first cannabinoid-based treatment to a first subject; receiving feedback from the first subject after the first cannabinoid-based treatment has been administered; adjusting the first cannabinoid-based treatment to provide a second cannabinoid-based treatment in response to the feedback; and administering the second cannabinoid-based treatment to the first subject.

In some embodiments, the first subject may be at least 65 years of age.

In some embodiments, the feedback may concern an ailment of aging.

In some embodiments, the second cannabinoid-based treatment may differ from the first cannabinoid-based treatment by an amount of at least one active ingredient.

In some embodiments, the amount of the at least one active ingredient in the second cannabinoid-based treatment may be reduced on a percentage weight basis compared to the first cannabinoid-based treatment.

In some embodiments, the amount of the at least one active ingredient of in the second cannabinoid-based treatment may be increased on a percentage weight basis compared to the first cannabinoid-based treatment.

In some embodiments, the second cannabinoid-based treatment may differ from the first cannabinoid-based treatment by a dosing regimen.

In some embodiments, a frequency of the first cannabinoid-based treatment may be greater than a frequency of the second cannabinoid-based treatment.

In some embodiments, a frequency of the second cannabinoid-based treatment may be greater than a frequency of the first cannabinoid-based treatment.

In some embodiments, a time of day for the first cannabinoid-based treatment may be earlier than a time of day for the second cannabinoid-based treatment.

In some embodiments, a time of day for the first cannabinoid-based treatment may be later than a time of day for the second cannabinoid-based treatment.

In some embodiments, a cannabinoid-based treatment (CBT) may include one or more cannabinoids as active ingredients. In some embodiments, the one or more cannabinoids may include cannabinol (CBN) as an active ingredient. In some embodiments, the one or more cannabinoids may include cannabidiol (CBD) as an active ingredient. In some embodiments, the one or more cannabinoids may include cannabichromene (CBC) as an active ingredient. In some embodiments, the one or more cannabinoids may include cannabigerol (CBG) as an active ingredient.

In some embodiments, the CBT may include from about 10 mg to about 200 mg of a cannabinoid. In some embodiments, the CBT may include a cannabinoid at a concentration from about 10 mg/mL to about 100 mg/mL if the cannabinoid is in a solution.

In some embodiments, the CBT may further include one or more additional active ingredients. In some embodiments, the one or more additional active ingredients may comprise theanine, melatonin, or a tea extract. In some embodiments, the tea extract may include one or more catechins. In some embodiments, the catechin may be epigallocatechin gallate (EGCG).

In some embodiments, the one or more additional active ingredients may include a local anesthetic. In some embodiments, the local anesthetic may include benzocaine, procaine, chloroprocaine, prilocaine, and lidocaine.

In some embodiments, the CBT may be flavored. In some embodiments, the flavor may be a fruit flavor. In some embodiments, the flavor may be a floral or an herbal flavor.

In some embodiments, the CBT may further include one or more sweeteners. In some embodiments, the one or more sweeteners may include a sugar.

In some embodiments, the CBT may include one or more excipients to solubilize the one or more cannabinoids or other components of the CBT. In some embodiments, the one or more excipients may include an oil or an alcohol.

In some embodiments, the CBT may include one or more additional ingredients. In some embodiments, the one or more additional ingredients may include coloring agents, thickeners, preservatives, aromatic agents, terpenes, oils, syrups, waxes, fats, and acids. In some embodiments, the one or more additional ingredients may be natural.

In some embodiments, the CBT may be in the form of a tincture.

In some embodiments, the CBT may be in a form for oral administration. In some embodiments, the CBT may be in the form of a gummy, lozenge, transdermal, or a vape.

In some embodiments, the CBT may be in a form for topical administration. In some embodiments, the CBT may be in the form of an oil, lotion, stick, or transdermal patch.

### BRIEF DESCRIPTION OF THE FIGURES

The accompanying drawings, which are incorporated herein and form a part of the specification, illustrate the aspects of the present disclosure and, together with the description, further serve to explain the principles of the aspects and to enable a person skilled in the pertinent art to make and use the aspects. The drawings are for illustration purposes only, show exemplary non-limiting embodiments, and are not necessarily drawn to scale.

FIG. 1A is a high-level conceptual diagram of one example of a system, in accordance with some embodiments;

FIG. 1B is a block diagram of one example of the functional components of the system illustrated in FIG. 1A, in accordance with some embodiments;

FIG. 1C is a block diagram of one example of the functional components of a computing device that may be used with the system illustrated in FIGS. 1A and 1B, in accordance with some embodiments;

FIG. 2 is a high-level flow diagram of one example of a user interacting with a hub network, in accordance with some embodiments;

FIGS. 3A-31 are examples of graphical user interfaces (GUIs) that may be presented to a subject during an enrollment/registration process in accordance with some embodiments;

FIGS. 4A-4L are examples of GUIs that may be presented to a subject to elicit feedback of one or more cannabinoid-based therapies (CBTs), in accordance with some embodiments;

FIG. 5 is an example of a GUI that may be presented to a subject showing one or more graphical interpretations of data collected from the subject over a period of time, in accordance with some embodiments;

FIG. 6 is a conceptual high-level diagram showing one example of an interaction between a subject and a hub network, which may result in a change in one or more CBTs for the subject, in accordance with some embodiments; and

FIG. 7 is a conceptual high-level diagram showing one example of an interaction between plural subjects and a hub network, which may result in a change in one or more CBTs for one or more of the subjects, in accordance with some embodiments.

### DETAILED DESCRIPTION

This description of the exemplary embodiments is intended to be read in connection with the accompanying drawings, which are to be considered part of the entire written description. As used herein, use of a singular article such as “a,” “an” and “the” is not intended to exclude pluralities of the article's object unless the context clearly and unambiguously dictates otherwise. The use of the singular includes the plural unless specifically stated otherwise. The use of “or” means “and/or” unless stated otherwise. Furthermore, the use of the term “including,” as well as other forms such as “includes” and “included,” is not limiting. In addition, terms such as “element” or “component” encompass both elements and components comprising one unit, and elements and components that comprise more than one subunit, unless specifically stated otherwise. Additionally, the section headings used herein are for organizational purposes only, and are not to be construed as limiting the subject matter described.

The following description is provided as an enabling teaching of a representative set of examples. Many changes can be made to the embodiments described herein while still obtaining beneficial results. Some of the desired benefits discussed below can be obtained by selecting some of the features discussed herein without utilizing other features. Accordingly, many modifications and adaptations, as well as subsets of the features described herein are possible and can even be desirable in certain circumstances. Thus, the following description is provided as illustrative and is not limiting.

Conventional medications, including opioids, are ill fitted to treat many of the prevalent age-related indications found in seniors (e.g., “ailments of aging”). Further, opioids are known to be highly addictive and excessive intake of such medicines may often result in medical dependencies and severe long-term health implications.

However, cannabinoid-based therapies (“CBTs”) provide a viable, cost-effective alternative to traditional therapies, including for seniors (e.g., individuals 65 years and older), which demographic outpaces other demographics and is expected to reach over 20 percent of the population by 2030. CBT research is still in its infancy, but preliminary research indicates that CBTs are particularly useful in treating a number of ailments of aging, including physical (e.g., chronic pain and arthritis in the hips, knees, and spine, for example), mental (e.g., dementia, Alzheimer's, sundowners, and other cognitive challenges, including depression, and anxiety and isolation), general pain relief and opioid dependency, and wellness (e.g., sleeping and agitation).

The inventor has spent considerable time and resources in developing systems and methods to improve the safety and efficacy of CBTs that also provide improved access to these vulnerable demographics. This groundbreaking work has resulted in one or more prototype systems and methods that facilitate the exchange and analysis of data leading to the development of new CBTs. By connecting researchers; patients (or subjects), which may be living by themselves, in a medical facility, and/or in a group home (e.g., a living laboratory); insurance providers, including Centers for Medicare and Medicaid Services (CMS) and the CMS Innovation Center (CMMI); and producers (e.g., cannabis growers and operators), the disclosed systems and methods enable the development and refinement of CBTs. In some embodiments, the disclosed systems and methods include the use of a real-world living laboratory in which the use of developmental CBTs are tracked. The systems or platforms may facilitate and/or enable telehealth so that otherwise isolated individuals may be provided with access to care. The disclosed systems and methods also may be configured to increase subject-knowledge of CBTs to improve subject understanding, awareness, and acquisition. In some embodiments, subjects may be connected seamlessly to one or more providers or prescribers of CBTs. In some embodiments, one or more insurance and/or government agencies may be provided with access to certain data to facilitate payment and reimbursement.

The disclosed systems and methods advantageously enable the collection of real-world evidence (“RWE”) (e.g., data regarding the usage, or the potential benefits and risks, of a drug derived from sources other than a traditional clinical trial) and/or real-world data (“RWD”) (e.g., data relating to a patient health status and/or the delivery of health care routinely collected from a variety of sources). In some embodiments, data from a clinical trial (e.g., a research study in which one or more human subjects are assigned to one or more interventions, including a placebo or other control, to evaluate the effects of those interventions on health-related biomedical or behavioral outcomes) may also be collected and/or analyzed along with the RWE and/or RWD. The data may be analyzed to evaluate the efficacy and/or safety of one or more CBTs.

As noted above, the disclosed systems and methods may be used to collect clinical data. This clinical data concerning one or more CBTs (or plant-based therapies) may include clinical data collected from Medicare (and other) beneficiaries and may be compared to traditional therapy methods. The comparisons may be used to facilitate multi-tier payer strategies by engaging existing CMS participants of value-based care demonstrations and to engage with Medicare Advantage Plans. Examples of such CMS participants include, but are not limited to, ACO Reach, Direct Contracting, Primary Care First, and Shared Savings.

In some embodiments, the data collected by the disclosed systems and methods may be used to develop new payment and service delivery models, such as through the CMMI. Examples of CMMI models that may be modified and/or used as a guideline include, but are not limited to, CMMI multi-payer comprehensive primary care demonstration model, CMMI direct contracting model, and CMMI primary care demonstration model. The disclosed systems and method also may be used to support research and development for CMMI with HIPAA-compliant patient and provider portals for data collection and analysis. Further, in some embodiments, the data may be analyzed to develop financial models for providing coverage for CBTs as well as to analyze the effectiveness of CBTs.

### System Overview

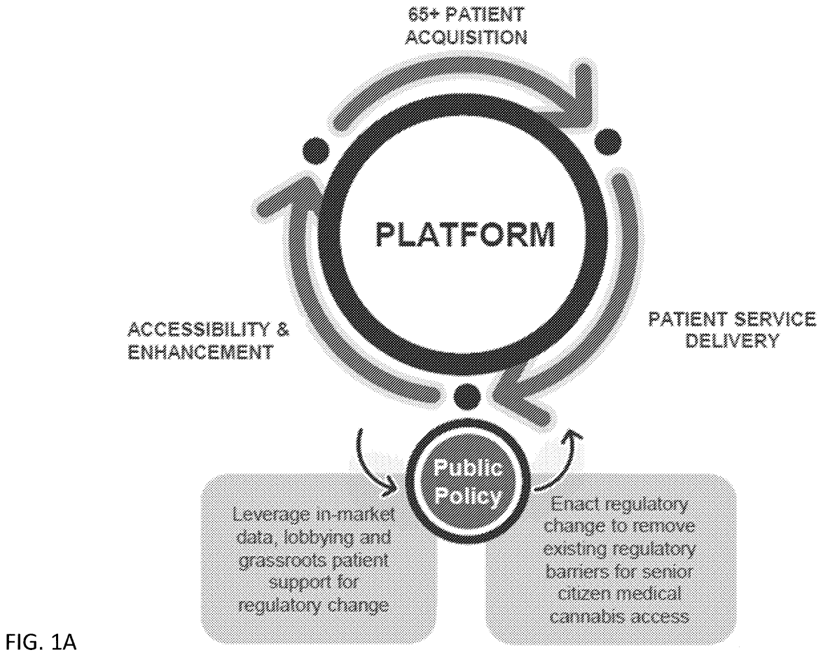

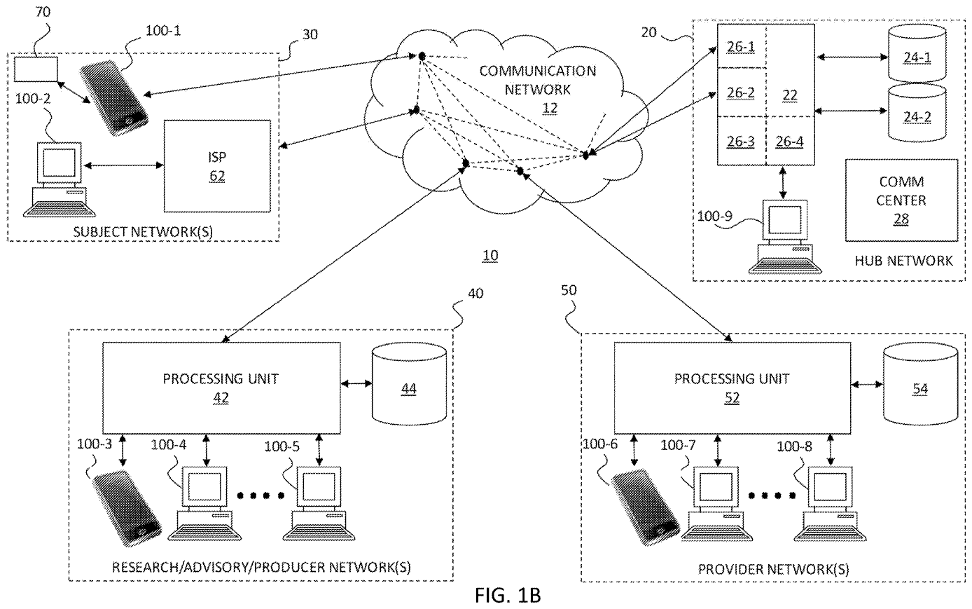

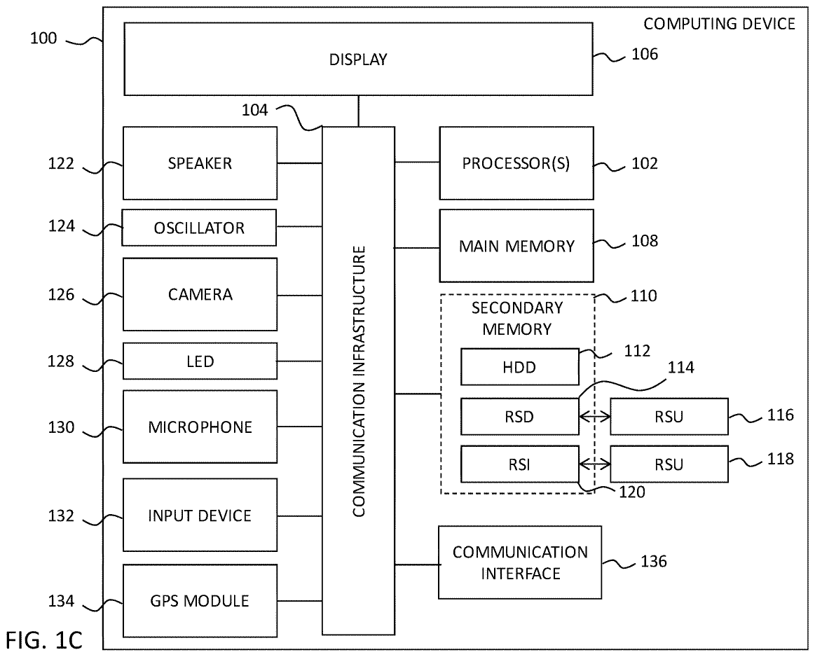

FIG. 1A is high-level conceptual diagram of one example of a system or platform that may be used to improve the efficacy of a treatment using a cannabis-derived substance in a vulnerable population in accordance with some embodiments. Conceptually, the platform or system may be considered to be the “hub” with one or more other interested parties (e.g., subjects or patients, medical providers, researchers, producers, insurance companies, and/or government agencies, to list only a few possible examples) communicatively coupled to the hub as “spokes.” In some embodiments, the spokes may be coupled communicatively to the hub via a network through which data may be transferred. The hub may be configured to collect, format, and/or analyze data to facilitate the development of one or more CBTs. The hub may further be configured to facilitate the requisite analysis needed for CBTs to be covered by one or more insurance providers, including Medicaid and/or Medicare.

FIG. 1B depicts one example of a system or environment 10 in accordance with some embodiments. The system 100 may include one or more networks, e.g., hub network(s) 20, subject network(s) 30, research and/or advisory board network(s) 40, provider network(s) 50, which are coupled via a communication network 12. Communication network 12 may be a wide area network (“WAN”), a local area network (“LAN”), personal area network (“PAN”), or other communication network as will be understood by one of ordinary skill in the art. In some embodiments, communication network 12 may be the Internet and computing devices 100 may be referred to as being online. For example, “online” may mean connecting to or accessing source data or information from a location remote from other devices or networks coupled to communication network 12.

In some embodiments, hub network 20 may include one or more servers that may be coupled, e.g., physically and/or communicatively, together. Each server may include one or more processing unit 22 coupled to one or more data storage units 24-1, 24-2 (collectively, “data storage units 24” or “data storage 24”). Data may be stored in the data storage units 24 in a structured or unstructured manner. For example, data may be stored in a database, such as a relational and/or sequential database, which may be referred to as a database management system (“DBMS”), or data may be stored in an object store and accessed using a hash table and/or an S3 protocol. The processing unit 24 may be configured to provide one or more front-end graphical user interfaces (“GUIs”) 26 (e.g., a subject GUI 26-1, a research and/or advisory board GUI 26-2, a provider GUI 26-3, and one or more back-end or administrative GUIs or portals 26-4). The GUIs 26 may be accessible to one or more computing devices 100, which may be locally or remotely located. The GUIs or portals 26 can take the form of, for example, a webpage that is displayed using a browser program executed by a computing device 100. In some embodiments, a GUI 26 may be displayed on a computing device 100 via a software application. For example, system 10 may include additional servers programmed or partitioned based on permitted access to data stored in data storage 24. As used herein, “portal” is not limited to general-purpose Internet portals, such as YAHOO!® or GOOGLE®, but also includes GUIs that are of interest to specific, limited audiences and that provide the party access to a plurality of different kinds of related or unrelated information, links and tools as described below. “Webpage” and “website” may be used interchangeably herein.

Hub network 20 may also include or be in communication with a communication center 28. The communication center 28 may be staffed with one or more communication center individuals and/or may include an automated communication center system. For example, the communication center 28 may be able to receive and respond to a variety of communications, including emails, text message, chats, and telephone calls, which may be received from subjects, medical providers, and/or other individuals. The communication center 28 may be configured to responding to one or more inquiries and/or questions that a subject, medical provider, and/or other individual may have, such as questions concerning registration/enrollment of a subject, a request for an appointment, a question concerning a prescribed or recommended, CBT, order fulfillment, and/or directions to a treatment center, to list only a few possibilities. In some embodiments, the communications may be transmitted to a subject, medical provider, and/or other individual in order to solicit information (e.g., concerning a subject's experience with one or more CBTs, as described herein) and/or to provide a reminder, such as indicating that a cannabis-derived substance to be used with one or more CBTs has been mailed or needs to be picked up, to list only a few possibilities. Information communicated to/from a subject, medical provider, and/or other individual may be stored in a data store 24. For example, a communication user may use a computing device, such as computing device 100-9 (or other computing device 100 not shown), to enter information into a data store 24. Additionally or alternatively, an automated communication system may use one or more voice-to-text and/or natural language processing techniques to extract relevant data from a communication (e.g., phone call, email, text message, chat, etc.) and store the information in a data store 24 as will be understood by one of ordinary skill in the art.

Subject network(s) 30 may include one or more networks of individuals who may be prescribed one or more CBTs by a medical professional and/or other individual. In some embodiments, a subject network may include a single user connected to communication network 12 via an Internet service provider (“ISP”) and/or through a cellular network connection. A subject may gain access to the hub network 20 using one or more computing devices, such as computing devices 100-1, 100-2. In some embodiments, a subject network 30 may include a plurality of individuals that may be grouped together or otherwise associated with one another. For example, in some embodiments, a subject network 30 may include a plurality of individuals living a common facility (e.g., an eldercare facility or living lab) or associated with one another, such as members of an organization like AARP, subscribers to a common insurance, and/or residents of a common jurisdiction (e.g., state, town, etc.), for example.

In some embodiments, subject network(s) 30 may include one or more sensors 70 that may be in communication with a computing device 100. Examples of the one or more sensors 70 include, but are not limited, a smart watch, wearable health monitor (e.g., FitBit), bed/mattress sensor, smart speaker (e.g., Echo Dot), biosensors, and/or medical equipment (e.g., sleep apnea machine (CPAP, BIPAP, etc.), heart monitor, implantable cardioverter defibrillator (ICD), etc.), to list only a few possibilities). As indicated in FIG. 1B, the one or more sensors 70 may be communicatively coupled to computing device 100 such that sensor data may be transmitted to hub network 20. It should be understood that although conceptually illustrated as communicating to hub network 20, the one or more sensors may transmit sensor data to hub network 20 without first transmitting the sensor data to computing device 100. The sensor data may include various types of data that may assist hub network 20 in determining an efficacy and/or safety of one or more CBTs, which may include the administration of one or more cannabis-derived substances. For example, a sensor 70 may transmit data concerning a subject's vital measurements (e.g., heart rate, breathing rate, blood oxygen level, blood pressure, etc.) and/or sleep habits (e.g., restlessness of sleep, number of times a patient got up during sleeping hours, etc.). As will be understood by one of ordinary skill in the art, the hub network 20 may include one or more identifiers (e.g., MAC address, IP address, serial number, etc.), which may be referred to as a “device ID,” in a profile associated with a subject such that the hub network 20 may store data received from the one or more sensors 70 in association with a subject. In some embodiments, a time stamp for a time at which the data was transmitted, received, and/or stored may also be stored by the hub network 20 in a data store 24.

Research/advisory/producer network(s) 40 may include one or more networks associated with one or more research and/or medical institutions. In some embodiments, a research/advisory/producer network 40 may include one or more processing units 42 and associated data storage 44. The one or more processing units 42 may be configured to operate as one or more servers, which may execute applications and/or store research data. In some embodiments, the one or more processing units 42 may be configured to access a remote data storage, such as a data storage 24 maintained by hub network 20. Researchers and/or medical professionals may gain access to communication network using a computing device (e.g., computing device 100-3, 100-4, 100-5) and via processing unit 42, as shown, although it should be understood that a researcher and/or medical professional may gain access to the hub network via a separate ISP (not shown) and/or cellular network, as will be understood by one of ordinary skill in the art.

As described herein, a researcher may be any individual that may be studying the effects, including mental and physical effects) of cannabis and/or other Schedule 1 narcotic on a person, the cultivation and/or manufacture of cannabis and/or other Schedule 1 narcotic, and/or other an individual providing other observational feedback. A medical advisory may be a researcher, doctor, policy maker, or other individual that may be provide advisory services concerning cannabis or other Schedule 1 narcotic. For example, the individual may be a member of an advisory board, which may be an advisory board for medical professionals that prescribe the use, dosage, and/or chemical makeup (e.g., amounts and/or ratios of active ingredient(s)). A producer user may be an individual associated with a grower of cannabis or other crop. In some embodiments, a producer user may be associated with an entity that manufactures or otherwise processes a raw material into a medicament.

One or more provider networks 50 may include one or more networks associated with one or more services providers. In some embodiments, a provider network may include one or more processing units 52 and associated data storage 54. The one or more processing units 52 may be configured to operate as one or more servers, which may execute applications and/or store service provider data. In some embodiments, the one or more processing units 52 may be configured to access a remote data storage, such as a data storage 24 maintained by hub network 20 and/or data storage 44 provided by research/advisory/producer network(s) 40. Service provider users, which may include one or more providers of medical, insurance, including Medicare and Medicaid, and/or other service provider professionals may gain access to communication network using a computing device (e.g., computing device 100-6, 100-7, 100-8) and via processing unit 52, as shown, although it should be understood that service provider professional may gain access to the hub network 20 via a separate ISP (not shown) and/or cellular network, as will be understood by one of ordinary skill in the art.

A computing device 100 (e.g., any of computing devices 100-1, 100-2, . . . , 100-8, etc.) may gain access to communication network 12 through an ISP 62 and/or through a wireless cellular communication network, a WAN hotspot, and/or through a wired or wireless connection with a computer as will be understood by one skilled in the art. Users, which may include one or more users associated with hub network 20 (which may include a communication center 28), patient network(s) 30, research/advisory/producer network(s) 40, and/or provider network(s), may use one or more computing devices 100 to gain access to hub network 20. Computing devices 100 may be any computing device capable of transmitting and receiving signals via a wired and/or wireless communication medium. Examples of client devices include, but are not limited to, mobile or cellular phones, smart phones, personal digital assistants (“PDAs”), laptop computers, tablet computers, music players, and e-readers, to name a few possible devices.

FIG. 1C is a block diagram of one example of an architecture of a computing device 100 in accordance with some embodiments. A computing device 100 may include one or more processors, such as processor 102. Processor(s) 102 may be any central processing unit (“CPU”), microprocessor, micro-controller, or computational device or circuit for executing instructions. Processor(s) 102 may be connected to a communication infrastructure 104 (e.g., a communications bus, crossover bar, or network). It should be apparent to one of ordinary skill in the art how to implement the method using computing devices 100 that include other systems or architectures beyond that set forth in FIG. 1C. Put another way, computing devices 100 may include some, all, or additional functional components as those of the computing device 100 illustrated in FIG. 1C.

Computing device 100 also may include a display 106 that may be configured to display graphics, video, text, and other data received from the communication infrastructure 104 (or from a frame buffer not shown) to a user. Examples of such displays 106 include, but are not limited to, LCD screens, LED displays, OLED display, capacitive touch screen, and a plasma display, to list only a few possible display types. Computing device 100 also may include a main memory 108, such as a random access (RAM) memory, and a secondary memory 110. Secondary memory 110 may include a more persistent memory such as, for example, a hard disk drive (“HDD”) 112 and/or removable storage drive (“RSD”) 114, which may represent a magnetic tape drive, an optical disk drive, solid-state drive (SSD), or other suitable memory. In some embodiments, removable storage drive 114 may read from and/or write to a removable storage unit (RSU) 116 in a manner that is understood by one of ordinary skill in the art. Removable storage unit 116 may represent a magnetic tape, optical disk, or the like, which may be read by and written to by removable storage drive 114. As will be understood by one of ordinary skill in the art, the removable storage unit 116 may include a tangible and non-transient machine-readable storage medium having stored therein computer software and/or data.

In some embodiments, secondary memory 110 may include other devices for allowing executable programs or other instructions to be loaded into computing device 100. Such devices may include, for example, a removable storage unit (RSU) 118 and a corresponding removable storage interface (RSI) 120. Examples of such units 118 and interfaces 120 may include a removable memory chip (such as an erasable programmable read only memory (EPROM)), programmable read only memory (PROM)), secure digital (SD) card and associated socket, and other removable storage units 118 and interfaces 120, which allow software and data to be transferred from the removable storage unit 118 to computing device 100.

Computing device 100 also may include a speaker 122, an oscillator 124, one or more cameras 126, a light emitting diode (LED) 128, a microphone 130, one or more input devices 132, one or more accelerometers (not shown), and a global positioning system (GPS) module 134. Examples of camera 126 features include, but are not limited to optical image stabilization (OIS), larger sensors, bright lenses, 4K video, optical zoom plus RAW images and HDR, and “Bokeh mode” with multi lenses and multi-shot night modes, to list a few possible examples. Camera 126 may comprise one or more lenses with different functions. By way of example, camera 126 may include an ultrawide sensor, telephoto sensor, time of flight sensor, macro sensor, megapixel (“MP”) sensor, and/or a depth sensor. As noted above, camera 126 is not limited to a single camera and includes a camera system that may include multiple different types of cameras, sensors, etc. By way of example, Apple's TrueDepth® camera system may include a front-facing “selfie” camera, an infrared emitter, an infrared camera, a proximity sensor, an ambient light sensor, a flood illuminator, and a dot projector that cooperate to obtain depth map and associated image. In other words, camera 126 of computing device 100 may have multiple sensors, cameras, emitters, or other associated components that work as a system to obtain image information for use by computing device 100.

Examples of an input device 132 may include, but are not limited to, a keyboard, buttons (physical or virtual), a trackball, voice input (e.g., microphone and corresponding software for recognizing speech), or any other interface or device through which a user may input data. In some embodiments, input device 132 and display 106 may be integrated into the same device. For example, display 106 and input device 132 may be touchscreen through which a user uses a finger, pen, and/or stylus to input data into computing device 100.

Computing device 100 also may include one or more communication interfaces 136, which allow software and data to be transferred between computing device 100 and external devices such as, for example, another computing device 100 or a server, which may be part of the hub network 20, research/advisory/producer network(s) 40, and/or provider network(s) 50. Examples of the one or more communication interfaces 132 may include, but are not limited to, a modem, a network interface (such as an Ethernet card or wireless card), a communications port, a Personal Computer Memory Card International Association (“PCMCIA”) slot and card, one or more Personal Component Interconnect (“PCI”) Express slot and cards, or any combination thereof. The one or more communication interfaces 136 may also include a wireless interface configured for short-range communication, such as near field communication (“NFC”), Bluetooth, or other interface for communication via another wireless communication protocol. As briefly noted above, one of ordinary skill in the art will understand that computing devices 100 may include some or all components illustrated in FIG. 1C.

Software and data transferred via the one or more communications interfaces 136 may be in the form of signals, which may be electronic, electromagnetic, optical, and/or other signals capable of being received by communications interfaces 136. These signals may be provided to communications interface 136 via a communications path or channel. The channel may be implemented using wire or cable, fiber optics, a telephone line, a cellular link, a radio frequency (RF) link, or other communication channels.

In this application, the terms “non-transitory computer program medium” and “non-transitory computer readable medium” refer to media such as removable storage units 116, 118, and/or a hard disk installed in hard disk drive 112. These computer program products may provide software to computing device 100 and/or other computing devices or processors. Computer programs (also referred to as “computer control logic”) may be stored in main memory 108 and/or secondary memory 110. Computer programs may also be received via the one or more communications interfaces 136. Such computer programs, when executed by a processor(s) 102, may enable the computing device 100 to perform the features of the methods and systems discussed herein.

In various embodiments, as shown in FIGS. 1B and 1C, computing device 100 may include a computing device such as a hashing computer, a personal computer, a laptop computer, a tablet computer, a notebook computer, a hand-held computer, a personal digital assistant, a portable navigation device, a mobile phone, a smart phone, a wearable computing device (e.g., a smart watch, a wearable activity monitor, wearable smart jewelry, and glasses and other optical devices that include optical head-mounted displays (OHMDs)), an embedded computing device (e.g., in communication with a smart textile or electronic fabric), or any other suitable computing device configured to store data and software instructions, execute software instructions to perform operations, and/or display information on a display device. Computing device 100 may be associated with one or more users (not shown). For example, a user may operate computing device 100 causing it to perform one or more operations in accordance with various embodiments.

In embodiments where some or all of the system 10 or methods performed by one or more components of the system 10, which may be partially or entirely implemented using software, the software may be stored in a computer program product and loaded into a computing device 100 and/or a server using removable storage drive, hard drive, and/or communications interface. The software, when executed by one or more processor, causes the one or more processor to perform the functions of the method described herein. In some embodiments, the methods may be implemented primarily in hardware using, for example, hardware components such as application specific integrated circuits (ASICs). Implementation of the hardware state machine to perform the functions described herein will be understood by persons skilled in the art. In some embodiments, the methods may be implemented using a combination of both hardware and software.

Embodiments of the subject matter described in this specification can be implemented in a system that includes a back end component, e.g., as a data server, or that includes a middleware component, e.g., an application server, or that includes a front end component (e.g., a computing device 100) having a graphical user interface or a Web browser through which a user can interact with an implementation of the subject matter described is this specification, or any combination of one or more such back end, middleware, or front end components. The components of the system can be interconnected by any form or medium of digital data communication, (e.g., a communication network 12). Communications network 12 may include one or more communication networks or media of digital data communication. Examples of communication network 12 include a local area network (LAN), a wireless LAN (e.g., a “WiFi” network), a RF network, a NFC network, a wireless Metropolitan Area Network (MAN) connecting multiple wireless LANs, NFC communication link(s), and a wide area network (WAN), e.g., the Internet and combinations thereof. In accordance with various embodiments, communications network 12 may include the Internet and any publicly accessible network or networks interconnected via one or more communication protocols, including, but not limited to, HyperText Transfer Protocol (HTTP), HyperText Transfer Protocol Secured (HTTPS), Secured Socket Layer/Transport Layer Security (SSL/TLS), and transmission control protocol/internet protocol (TCP/IP). Communications protocols in accordance with various embodiments also include protocols facilitating data transfer using radio frequency identification (RFID) communications and/or NFC. Moreover, communications network 12 may also include one or more mobile device networks, such as a GSM, PCT, LTE, and/or 5G network, for example, allowing a client device to send and receive data via applicable communications protocols, including those described herein.

A computing device 100 and a server, such as a server that is part of hub network 20, research/advisory/producer network(s) 40, and/or provider network(s) 50, may be generally remote from each other and typically interact through a communication network 12. The relationship of computing device 100 and a server may arise by virtue of computer programs running on the respective system components and having a client-server relationship to each other. System 10 may include one or more web/application servers (not shown) in embodiments used to gain access to many services provided by one or more of the network(s) 20, 40, 50.

In some aspects, a computing device 100 may store in memory one or more software applications that run on the computing device when executed by the one or more processors 102 of the computing device 100. In some instances, each computing device 100 may store software applications that, when executed by one or more processors 102, perform operations that establish communications with a server of one or more of the networks 20, 40, 50 (e.g., across communication network 12 via communication interface 136) and that obtain, from the server of one or more of the networks 20, 40, 50, information or data from a data store 24, 44, 54 in accordance with various embodiments.

In various embodiments, computing device 100 may execute stored software application(s) to interact with a server of one or more of the network(s) 20, 40, 50 via a network connection. The executed software applications may cause computing device 100 to communicate information (e.g., age, gender, race, prescription information, dosing regimen, location, and query answers, as described herein, to list only a few possibilities). As described below, executed software applications(s) may be configured to allow a user associated with computing device 100 to input data, which is transmitted to hub network 20. Further, hub network 20 may be configured to transmit data stored in a data storage 24 to a computing device 100 and/or to other network(s) 40, 50. Stored software application(s) on computing device 100 may be configured to access webpages on the Internet or other suitable network based communication capable of interacting with communication network 12, as will be understood by one of ordinary skill in the art. For example, a user may access a server of a network 20, 40, 50 via an Internet webpage. The server may be configured to render the Internet webpage for the user on computing device 100. Alternatively or additionally, a server may provide information to stored software application(s) on computing device 100 via communication network 12. The computing device 100 may display information provided by a server using a stored software application(s) graphical user interface display. A respective user account may be associated with a subject user, a researcher, medical professional, service provider, and/or other user as will be understood by one of ordinary skill in the art and described below.

As noted above, system 10 may include data stores 24, 44, 54, which may be configured to store and manage data, such as user account information, research data, subject health records, medical study data, real-time observation data collected from one or more subjects, and insurance data, to list only a few possibilities. The data stores 24, 44, 54 may be communicatively coupled with various modules and engines as will be understood by one of ordinary skill in the art.

It should be understood that various forms of data storage or repositories can be used in system 10 that may be accessed by a computing system, such as hard drives, tape drives, flash memory, random-access memory, read-only memory, EEPROM storage, in-memory databases like SAP HANA, and so on, as well as any combination thereof. Stored data may be formatted within data stores in one or more formats, such as flat text file storage, relational databases, non-relational databases, XML, JSON, blobs, objects, comma-separated values, Microsoft Excel files, or any other format known to those of ordinary skill in the art, as well as any combination thereof as is appropriate for the particular use. Data stores may provide various forms of access to the stored data, such as by file system access, network access, S3, a SQL protocol (e.g., ODBC), HTTP, FTP, NES, CIFS, and so on, as well as any combination thereof.

According to various embodiments, a computing device 100 may be configured to access a data storage 24, 44, 54 via a server associated with one or more networks 20, 40, 50. In various embodiments, a data storage 24, 44, 54 may be configured to maintain a database schema. For example, a database schema may be arranged to maintain identifiers in columns within data storage 24, 44, 54. In this aspect, identifiers may refer to specific information pertaining to one or more categories, such as those described herein. A database schema within a data storage 24, 44, 54 may be arranged or organized in any suitable manner within the system. Although examples of categorical identifiers are described herein, any number of suitable identifiers may be used to maintain records associated with the system described herein. In addition, a database schema may contain additional categories and identifiers not described above for maintaining record data in system 10. In some embodiments, a database schema may advantageously organize identifiers in a way that permits the system to operate more efficiently. In some embodiments, categories of identifiers in the database schema may increase efficiency by grouping identifiers.

In some embodiments, data may be stored as a block or blob in an object store that is accessible using one or more identifiers. For example, the object may include various types of information associated with a user and may be accessed using a hash or other unique (or substantially unique) value. One of ordinary skill in the art will understand that the data storage 24, 44, 54 may be configured in a number of ways to facilitate data storage and retrieval.

In various embodiments, a server may include computing components configured to store, maintain, and generate data and software instructions. For example, a server may include or have access to one or more processor(s) 24, 42, 52 and tangible, non-transitory memory devices for storing software or code for execution and/or additional data stores. Servers may include one or more computing devices configured to execute software instructions stored on to perform one or more processes in accordance with various embodiments. In some embodiments, data storage 24, 44, 54 may include or be associated with a server that executes software instructions to perform operations that provide information to at least one other component of computing environment 10 through a network, such as a communication network 12.

As noted above, a server, such as the server of hub network 20, may be configured to provide one or more websites, digital portals, or any other suitable service 26 that may be configured to perform various functions of hub network 20. In some embodiments, the one or more servers of hub network 20 may maintain one or more application programming interfaces (“APIs”) through which the functionality and services provided by server may be accessed through one or more application programs executed by a computing device 100. In various embodiments, the one or more servers of hub network 20 may provide information to software application(s) on computing 100 for presentation on a display 106. In some embodiments, one or more servers of hub network 20 may provide information to a computing device 100 (e.g., through the API associated with the executed application program), and computing device 100 may present portions of the information to corresponding users through a display 106.

In various embodiments, the hub network 20 may be configured to provide and/or receive information associated with services provided by other network(s) 30, 40, 50. For example, a computing device 100 may receive information from a server associated with hub network 20 via communication network 12, which may be stored in a locally accessible storage device and/or network-accessible storage devices and data stores (e.g., cloud-based storage). For example, a computing device 100 may execute stored instructions (e.g., an application program, a web browser, and/or a mobile application) to process portions of stored data and render portions of the stored data for presentation to the respective user or users. Hub network may include one or more servers, which may be incorporated as a corresponding node in a distributed network or as a corresponding networked server in a cloud-computing environment. Furthermore, servers may communicate via communication network 12 with one or more additional servers (not shown), that may facilitate the distribution of processes for parallel execution by the additional servers.

### Overview of Processes

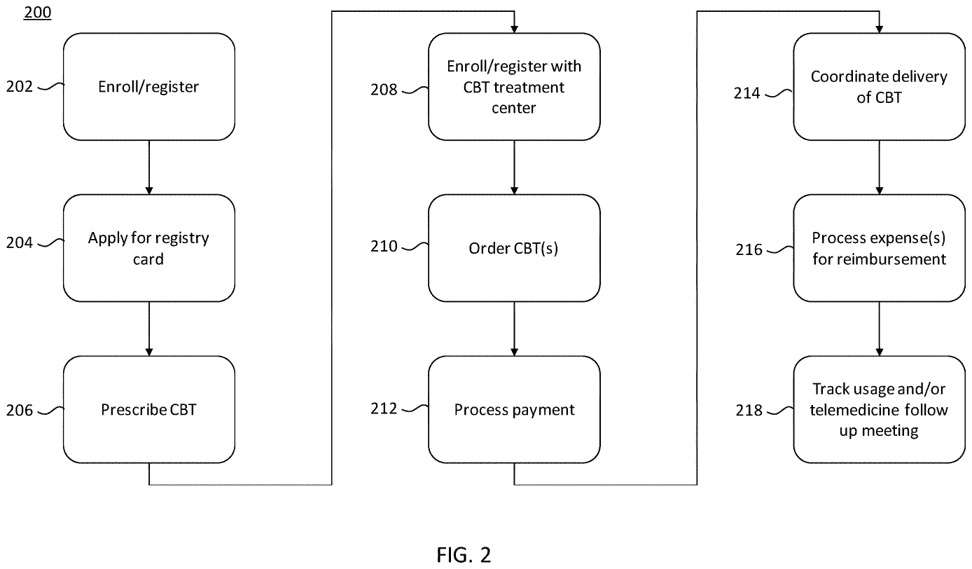

FIG. 2 is a high-level block diagram of one example of a process flow in accordance with some embodiments. The process 200 illustrated in FIG. 2 is one example of a general process through which a subject user interacts with a hub network 20. However, other processes may be performed as will be understood by one of ordinary skill in the art.

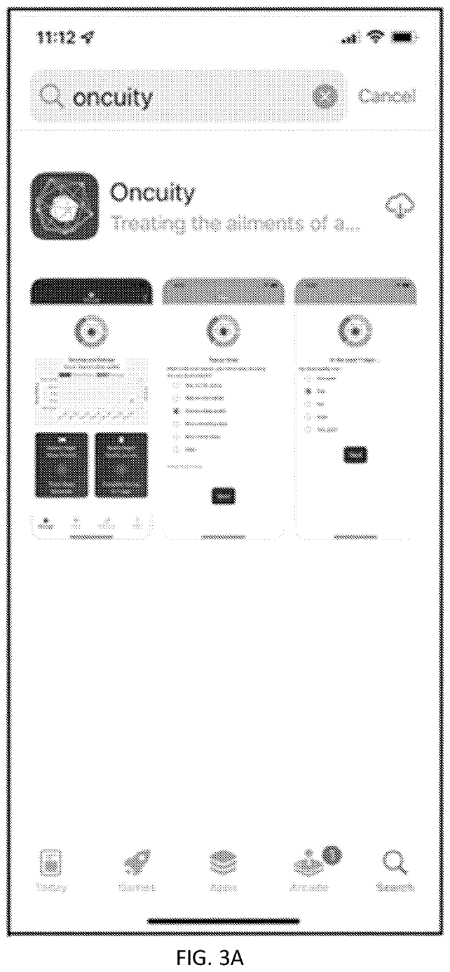

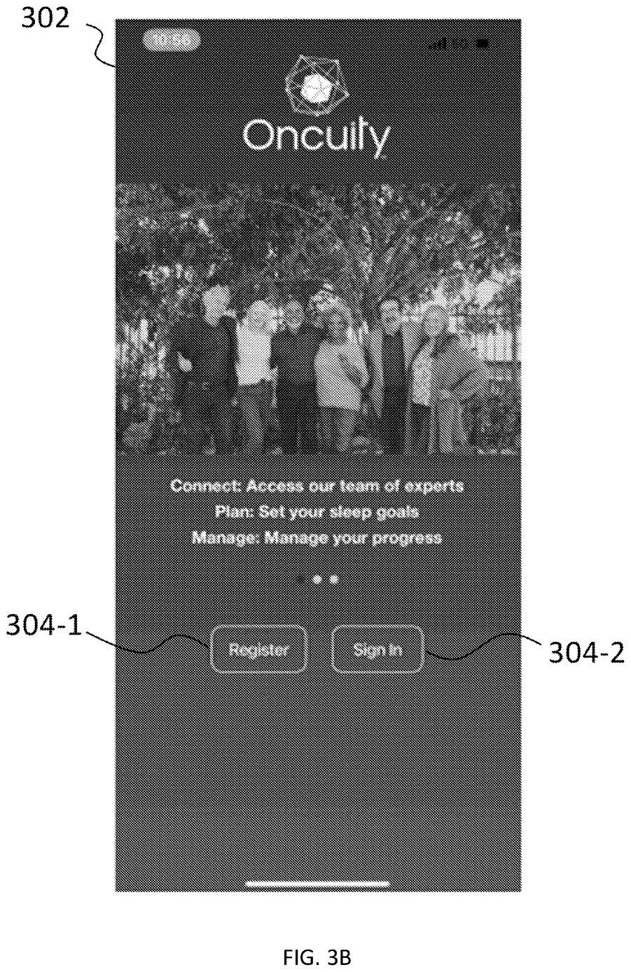

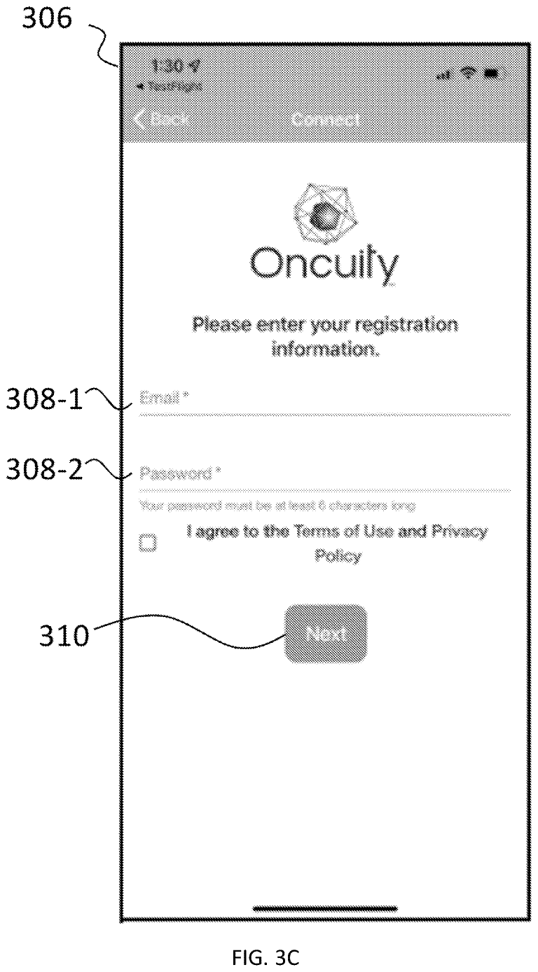

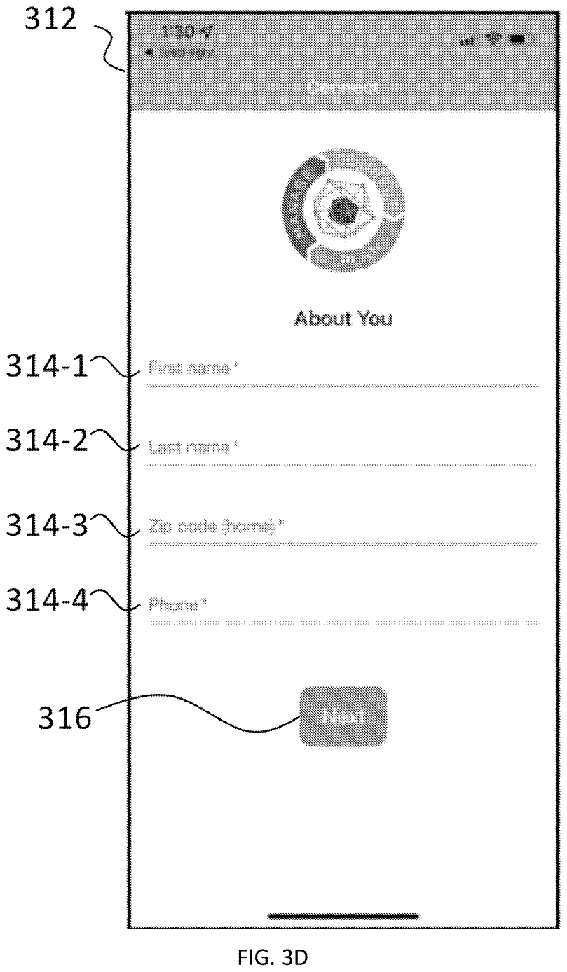

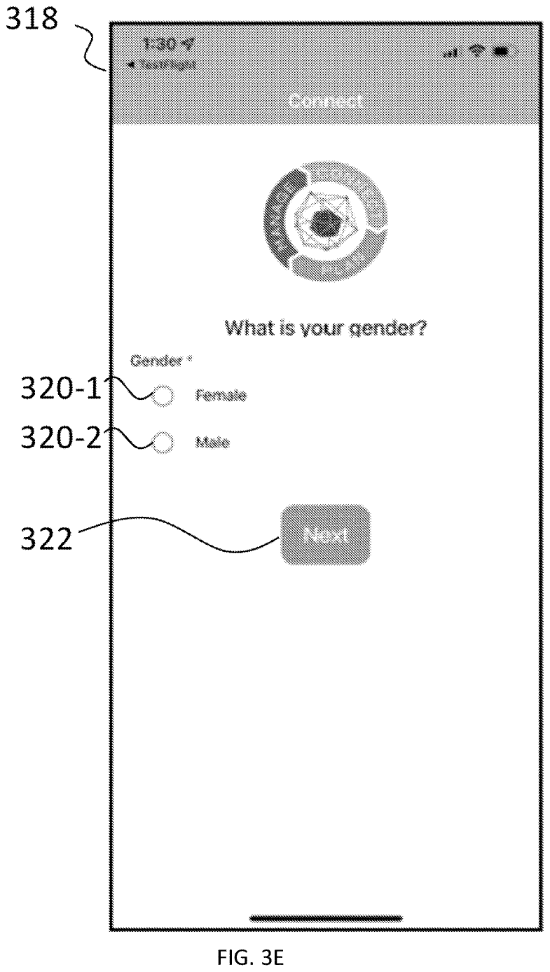

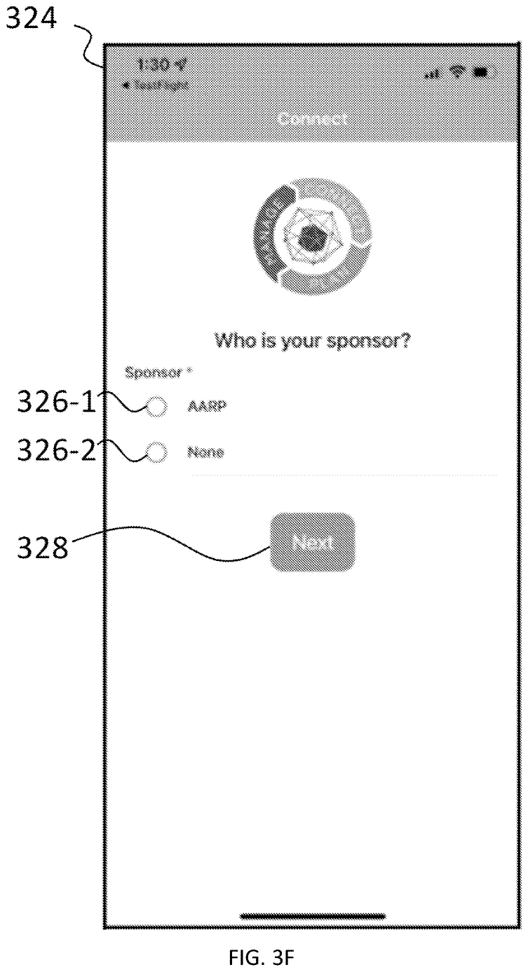

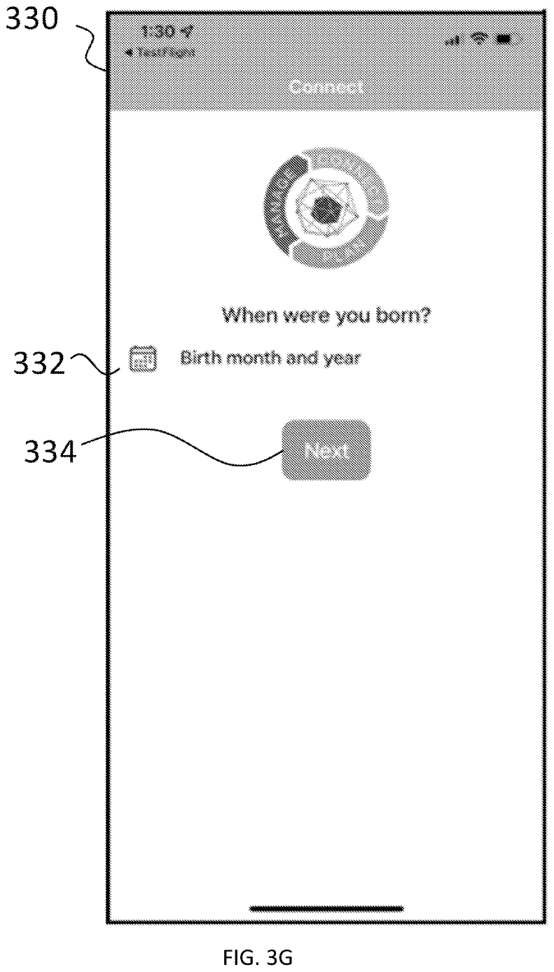

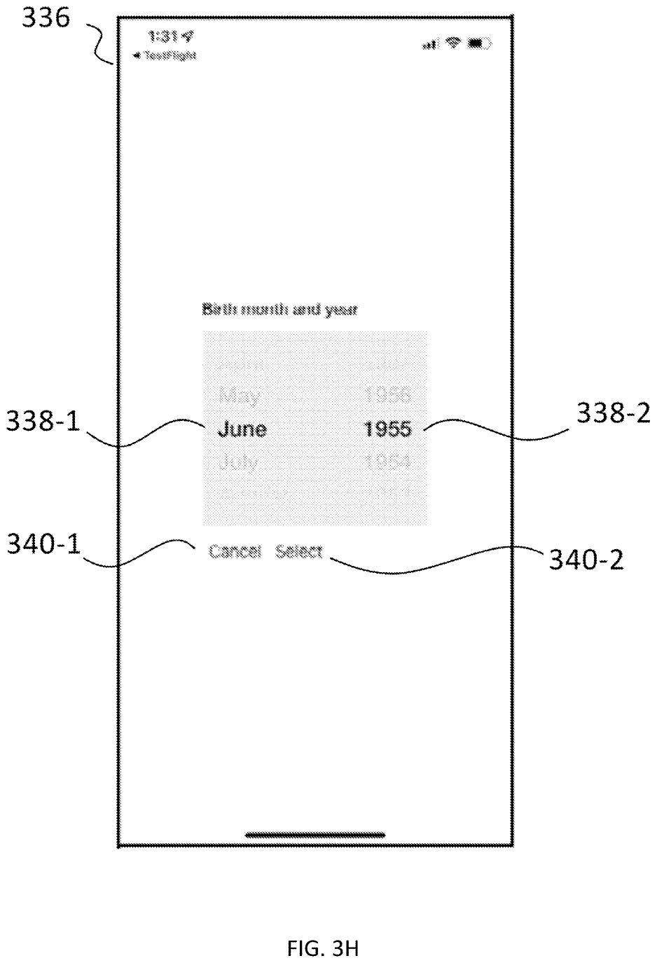

At block 202, a user may be enrolled or registered with the hub network 20. In some embodiments, enrollment may include creating a username and password. For example, a user may be enrolled or registered with hub network by navigating to a webpage maintained by hub network 20 using a computing device 100. Additionally or alternatively, a subject user may download a mobile application for installation on a mobile device, as will be understood by one of ordinary skill in the art. FIG. 3A illustrates one example of an app, named “Oncuity” in FIG. 3A, being made available in an app store, such as Apple's App Store and Google's Play app store, to list only a few examples. Upon installation, the user may be prompted to register/enroll with the hub network 20 by creating a username and password, such as shown in the GUI 302 in FIG. 3B. GUI 302 may be presented to a user on a display 106 of a computing device 100 as will be understood by one of ordinary skill in the art. As shown in FIG. 3B, the GUI 302 may include a virtual button 304-1 to “Register” and another virtual button to “Sign In” if the user has already registered and enrolled.

FIG. 3C illustrates one example of a GUI 306 that may be presented to a user upon the user selecting Register on GUI 302, such as by using an input device 132. As shown in FIG. 3C, the user may be prompted to enter an email address in entry field 308-1 and a password in entry field 308-2. An input device 132 may be used to enter the requested information into the entry fields 308, as will be understood by one of ordinary skill in the art. A virtual button 310 may be selected once the user has entered the email address and password in entry fields 308. In some embodiments, biometric information (e.g., fingerprint, facial recognition, voice recognition) may be collected and used to register the subject with hub network 20.

Additional information may be collected during the enrollment or registration. For example, a subject user's name, date of birth, social security number, address, phone number, physical address, email address, gender, marital status, ethnicity, household income, education level, and health information (including prescriptions (e.g., blood thinners, statins, undergoing chemotherapy, bronchodilator, cholesterol medication, antidepressants, pain relievers, cannabis, cannabinol, etc.), indications, ailments (e.g., sleep apnea, substance abuse, cancer, high blood pressure, diabetes, arthritis, Crohn's disease, anxiety or depression, epilepsy, glaucoma, insomnia, joint pain or inflammation, migraines, PTSD, Alzheimer's, schizophrenia, psychosis, COPD, etc.), to list only a few possibilities. General or subject health related questions may also be presented, including whether a subject experiences one or more of the following: trouble falling asleep, trouble staying asleep, dizziness, unsteadiness, heartburn or indigestion, panic attacks, and/or allergies (food, pet, other), for example.

In some embodiments, either upon completion or during enrollment, a subject's medical record may be associated with the subject's account or profile maintained by the hub network 20. As described above, hub network 20 may communicate with one or more other networks, including a medical provider network 50, which may include a medical provider network 50 for a medical provider at which the subject is a patient. Consequently, in some embodiments, the hub network 20 may be configured to access a medical record of a subject (assuming a subject agrees to such access), which may include additional medical information concerning a subject. Examples of such additional medical information includes, but is not limited to, DNA, RNA, proteins, metabolomics, lipids, metals, toxins, and immune-signatures, which may be identified in one or more tests previously performed on a subject, to list only a few possibilities. In some embodiments, the medical provider network 50 may also include or be communicatively coupled to an insurance provider network 50, such as private or government insurance provider network. The additional medical information may also include insurance coverage information as will be understood by one of ordinary skill in the art.

FIG. 3D illustrates an example GUI 312 including entry fields 314-1, 314-2, 314-3, 314-4 (collectively, “entry fields 314”). In some embodiments, entry field 314-1 provides a field in which the user will enter the user's first name, entry field 314-2 provides a field in which the user will enter the user's last name, entry field 314-3 provides a field in which the user will enter the user's zip code, and entry field 314-4 provides a field in which the user will enter the user's phone number. A user may enter the requested information, which may include text and numbers, using an input device 132 of a computing device 100 as will be understood by one of ordinary skill in the art. GUI 312 may also display a virtual button 316 labeled “Next” that may be pressed when the user has entered the requested information. Pressing the “Next” button 316 may take the user to a next page of the registration process.

FIG. 3E illustrates another example of a GUI 318 that may be presented to a subject user on a display 106 of a computing device 100 during the registration process, in accordance with some embodiments. As shown in FIG. 3E, GUI 318 may display the question “What is your gender?” and provide one or more radio buttons 320-1, 320-2 for selection by a subject user. It should be understood that while only two radio buttons are shown, other radio buttons may be provided and/or the radio buttons may be replaced with other input mechanisms, such as an entry field. GUI 318 may also include a virtual button 322 labeled “Next” that may take a user to another GUI as part of the registration process.

In some embodiments, a subject user may be queried as to whether they have a sponsor. For example, FIG. 3F illustrates one example of a GUI 324 querying the user to identify a sponsor, such as AARP. Although only AARP is shown in FIG. 3F, it should be understood that other organizations or entities may be presented. A user may select one or more sponsors by selecting the one or more radio buttons 326-1, 326-2 presented in the GUI 324 as will be understood by one of ordinary skill in the art. GUI 324 may include a virtual button 328 labeled “Next” that may be configured to cause another GUI to be displayed to the user in response to be pressed.

FIG. 3G illustrates another example of a GUI 330 that may be presented to a user during a registration process in accordance with some embodiments. GUI 330 may request the user identify the month and year in which the user was born. In some embodiments, the GUI 330 may include a virtual button 332 representing a calendar that, when pressed, may cause another GUI, such as GUI 336 shown in FIG. 3H, to be displayed. The GUI 336 may include list selection buttons 338-1, 338-2, with one list selection button 338-1 providing for the selection of a month and another button 338-2 providing for the selection of a year. GUI 336 may also include a virtual button 340-1 labeled “Cancel” and a virtual button 340-2 labeled “Select.” If the button 340-1 is selected, then the user may be taken back to GUI 330 without having entered month and year of birth. If the button 340-2 is selected, then the user may be taken back to GUI 330 with the month and year of birth having been recorded. As shown in FIG. 3G, GUI 330 may include a virtual button 334 labeled “Next” that may be selected by the user when the user has entered a birth month and year. In some embodiments, the user may be queried for a day of birth. In some embodiments, the virtual button 334 may be greyed out or unable to be selected until the user enters a date of birth as will be understood by one of ordinary skill in the art. The information input by the user may be stored locally on the computing device 100, such as in main memory and/or secondary memory 110. Alternatively or additionally, the information input by a user may be stored remotely from the computing device, such as in a data store 24 of the hub network 20. As will be understood by one of ordinary skill in the art, the data store 24 may store the information input by the user during the registration process in an object or in a plurality of fields or categories of a database. The stored data may be associated with a profile of the user, which may be indexed by a hash or other unique identifier. In some embodiments, the input data is stored in a HIPAA compliant manner.

In some embodiments, a subject user may have a question about the enrollment process, CBTs, and/or want to speak with a counselor. FIG. 31 illustrates one example of a GUI 342 that may be accessible during the registration/enrollment process. As shown in FIG. 31, a virtual button 344 labeled “Connect” may be provided during the registration process and pressing and/or otherwise selecting the virtual button 344 may result in the GUI 342 being presented to a subject, such as on a display 106 of a computing device 100. The GUI 342 may include one or more virtual buttons or hyperlinks 346-1, 346-2 (collectively, “virtual buttons 346” or “hyperlinks 346”) that may be associated with different actions or GUIs. For example, virtual button or hyperlink 346-1, which may be associated with the text “Call an Oncuity Counselor,” may display a phone number, activate a phone of the computing device, and/or automatically place a call to a counselor, such as a counselor located at communication center 28 of hub network 20, shown in FIG. 1, when pressed or otherwise selected. Virtual button or hyperlink 346-2, which may be associated with the text “Email an Oncuity Counselor,” may display an email address, activate an email application running on the computing device, and/or cause an email to be sent automatically when pressed or otherwise selected. Although not shown expressly in FIG. 31, it should be understood that other communication methods or processes may be provided or made accessible, such as text messaging (e.g., virtual chat) and/or video calling, to list only a couple of possibilities. As described above, the communication center 28 may include one or more individuals and/or an automated communication system to respond to inquiries from one or more subjects. Information exchanged during the communication (e.g., email, text, chat, phone call, etc.) may be stored in one or more data stores 24.

In some embodiments, the registration or enrollment process may query the user for additional information, such as information concerning user's health (e.g., physical and/or mental) or wellness (e.g., sleep). For example, one or more GUIs may be provided to a user, such as on a display 106 of a computing device, and present one more questions to the user concerning the user's health and wellness. Examples of such questions include, but are not limited to, reasons for being interested in one or more CBTs, prior experience with CBTs, and existing medications and/or treatments for maladies. In some embodiments, the user may be presented with the questions before the completion of the registration process and a user is prescribed or recommended a CBT, which may include the administration of (e.g., ingestion, topical application, etc.) one or more cannabis-derived substances. However, in some embodiments, the user may have been recommended one or more CBTs prior to completing the registration process.

Referring again to FIG. 2, at block 204, the hub network 20 may facilitate the application for a registry card for a subject user. In some embodiments, the hub network 20 may be configured to coordinate the application based on information obtained during the registration processor at block 202. For example, the hub network 20 may be configured to analyze data input by the subject user, including name, address, date of birth, and medical information and/or interest in one or more CBTs, and establish communication with another network, such as one of the provider network(s) 50 that is licensed to supply or prescribe CBTs in the same jurisdiction in which the subject user resides. In some embodiments, the hub network 20 generate and transmit an electronic message to one or more of the provider network(s) 50 requesting a physician or other prescribing professional to assist in the application. Depending on the jurisdiction and legal requirements, a telehealth meeting between the subject and physician may be required, and the hub network 20 may coordinate such an appointment. In some jurisdictions, a telehealth meeting may not be required, and the medical provider may be able to prescribe or sign off on the application for the subject user by either entering information in the provider network 50, which information is transmitted to the hub network 20, or by logging directly into the hub network 20 and completing the paperwork mandated by the jurisdiction for the subject to obtain a registry identification (“ID”) card.

At block 206, one or more CBTs may be prescribed. In some embodiments, one or more CBTs may be prescribed during the same transaction as applying for a registry ID card. Accordingly, the same physician or prescribing professional may issue a script for one or more CBTs. As will be understood by one of ordinary skill in the art, the one or more CBTs may be prescribed based on data obtained from the subject user, such as data entered into the app during the registration or registration process and/or during the telehealth meeting. In some embodiments, the hub network 20 may be configured to suggest one or more CBTs to the physician or prescribing professional based on the data input by the subject user during the registration process. For example, a data store 24 may include one or more rules or machine learning algorithms configured to be executed by the one or more processors 22 of the hub network 20. The rules and/or machine learning algorithms may be applied to the data stored in the one or more data stores 24 associated with the subject user and one or more suggested CBTs may be determined based on the analysis. The suggested CBT may include a product type (e.g., one or more CBTs configured to alleviate one or more symptoms and/or side effects associated with aging), a dosage amount, and/or a dosage schedule or regimen. The recommendation for one or more CBTs may be transmitted from the hub network 20 to the computing device used by the physician or prescribing professional, such as a computing device 100-3, 100-4, . . . , 100-8, for example.

At block 208 in FIG. 2, a recommendation or identification of one or more CBT treatment centers may be made for the subject. In some embodiments, the CBT treatment center(s) may be the CBT treatment center(s) with which the prescribing physician or professional is associated. However, it should be understood that the CBT treatment center(s) recommended and/or identified at block 208 may be different from any CBT treatment center(s) with which the prescribing physician or professional is associated. Hub network 20 may coordinate the enrollment of the subject user as a subject of the one or more CBT treatment center(s), such as by communicating with one or more provider network(s) 50 via communication network 20.

At block 210, one or more CBT(s) may be ordered for the subject. In some embodiments, the order may be placed by the hub network 20 automatically in response to the one or more CBTs being prescribed at block 206 and/or the subject user being enrolled and/or registered with a CBT treatment center at block 208. For example, the processor(s) 22 of hub network 20 may execute a routine or process in which an order is placed with one or more producer networks 40. As will be understood by one of ordinary skill in the art, the hub network 20 and one or more producer networks 40 may communicate via communication network 12 to facilitate the transaction. In some embodiments, the transaction is automatically performed by the hub network 20. However, in some embodiments, the hub network 20 may be configured to facilitate the order with input from one or more users, such as input from a prescribing profession and/or a subject user.

At block 212, the payment for the order of the CBT(s) may be placed. It should be understood that the process performed at block 212 may be performed as part of the process performed at block 210, in some embodiments. The payment for the CBT(s) may be made using a credit card, via automated clearinghouse (“ACH”), or via any other suitable payment transaction method as will be understood by one of ordinary skill in the art. The hub network 20 may obtain a subject user's credit card and/or bank account information as part of the registration and/or enrollment performed at block 202, or the hub network 20 may query the subject user for the payment information at block 212. For example, the one or more processors 22 of hub network 20 may cause a request for the payment information to be transmitted to a computing device associated with the subject, such as computing device 100-1, 100-2. A notification may be presented to the user, such as on display 106, via speaker 122, via oscillator 124, and/or via LED 128. The visual, audible, and/or tactile notification may alert the user to input the requested payment information, such as via an app running on the computing device 100. The user may enter the requested payment information using an input device 132 and the payment information may be transferred from the computing device 100 to hub network 20 where it may be stored in a data store 24.

At block 214, delivery of the one or more CBT(s) may be coordinated. For example, the hub network 20 may provide the address for the subject to the one or more producer networks 40 from which the CBT(s) have been ordered. In some embodiments, the subject user may be queried as to the type of delivery (e.g., standard, next day, via courier or third party delivery service, etc.) that is requested for the order. The subject user may be queried in a similar way in which the subject user is queried to provide payment information, as described above with respect to block 212. As will be understood by one of ordinary skill in the art, the coordination of the delivery may be performed during a same or different transaction from the payment transaction.

At block 216, the hub network 20 may be configured to process expense(s) for reimbursement. For example, the hub network 20 may be configured to submit the necessary paperwork and/or information to a private insurance company and/or government entity, such as Medicare and/or Medicaid. Hub network 20 may facilitate the reimbursement process by one or more processors 22 communicating with one or more provider networks 50, such as a private insurance provider, including a government insurance provider (e.g., Medicare/Medicaid), Hub network 20 may cause the payment amount, script information, subject information, and/or any other necessary information to be transmitted to the relevant one or more provider networks 50 to facilitate reimbursement as will be understood by one of ordinary skill in the art.

At block 218, the usage and effectiveness of the one or more prescribed CBT(s) may be tracked. In some embodiments, the usage and effectiveness of the CBT(s) is tracked by performing one or more telehealth calls or meetings, which may be facilitated using an app running on the computing device 100 of the subject and medical provider as will be understood by one of ordinary skill in the. In some embodiments, the communication center 28 of the hub network 20 may be configured to solicit usage and experience information from one or more subjects. For example, the communication center 28 may be configured to communicate with one or more subjects via telephone, email, text, chat, and/or through other suitable means as described above. The communication center 28 may include one or more individuals and/or an automated communication system to query one or more subjects concerning about the experience a subject is having with one or more CBTs periodically. Responses to the queries, examples of which are described below and elsewhere herein, may be extracted from a subject's response and stored in a data store 24. For example, a communication system individual may record a subject's response(s) to one or more questions into an electronic form presented on a display 106 on a computing device 100 used by the communication system user. In some embodiments, an automated communication system may use one or more voice-to-text and/or natural language processing techniques to extract relevant data from a communication (e.g., phone call, email, text message, chat, etc.) and store the information in a data store 24 as will be understood by one of ordinary skill in the art. In some embodiments, a subject may respond to questions presented by an automated communication system by pressing one or more buttons on a telephone (e.g., 0, 1, 2, etc.) and the automated communication system may store the response in a data store 24. Solicitations/queries may be sent (e.g., through a telephone call, email, text, chat request, etc.) periodically, such as daily, bi-weekly, weekly, bi-monthly, monthly, quarterly, semi-annually, etc. It should be understood that solicitations/queries for different types of information may be sent on different timelines, as first information may be queried on a first schedule (e.g., daily), second information may be queried on a second schedule (e.g., weekly), third information may be queried on a third schedule (e.g., bi-monthly), and so forth. One of ordinary skill in the art will understand that other schedules may be used.

Alternatively or additionally, the tracking may be performed by the subject user with the assistance of the app running on the computing device 100 of the user. For example, a user may log or sign into the app with which the user previously registered or enrolled at block 202. In some embodiments, the user may press the “Sign In” virtual button 304-2 on the GUI 302 shown in FIG. 3B.

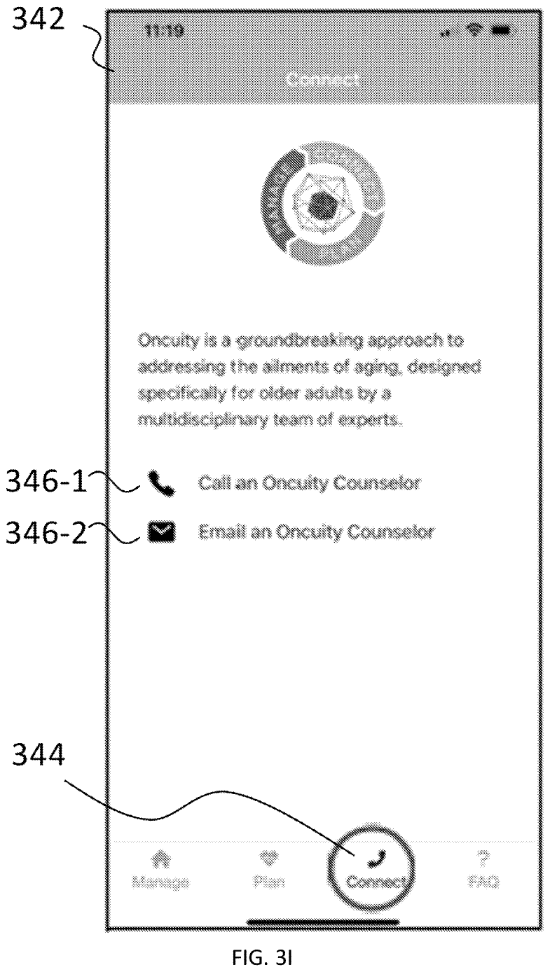

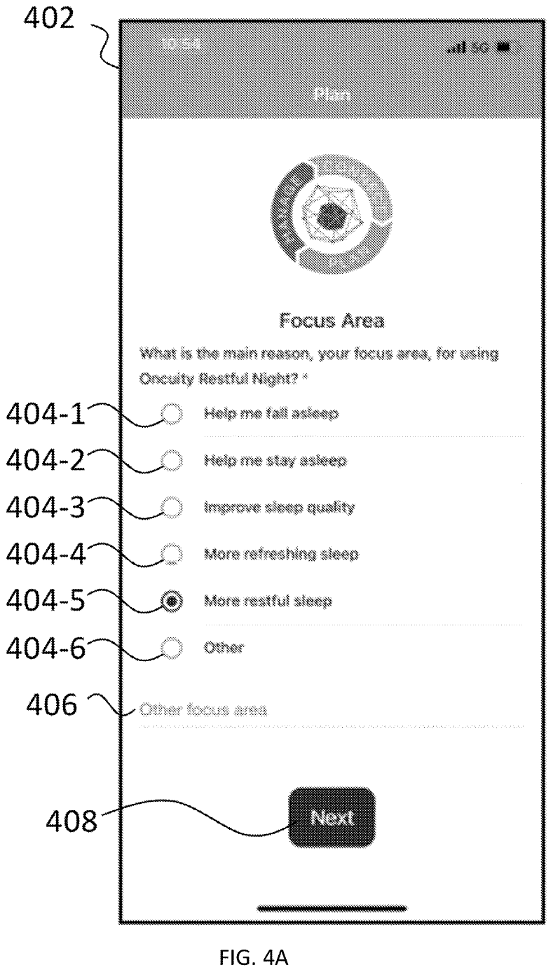

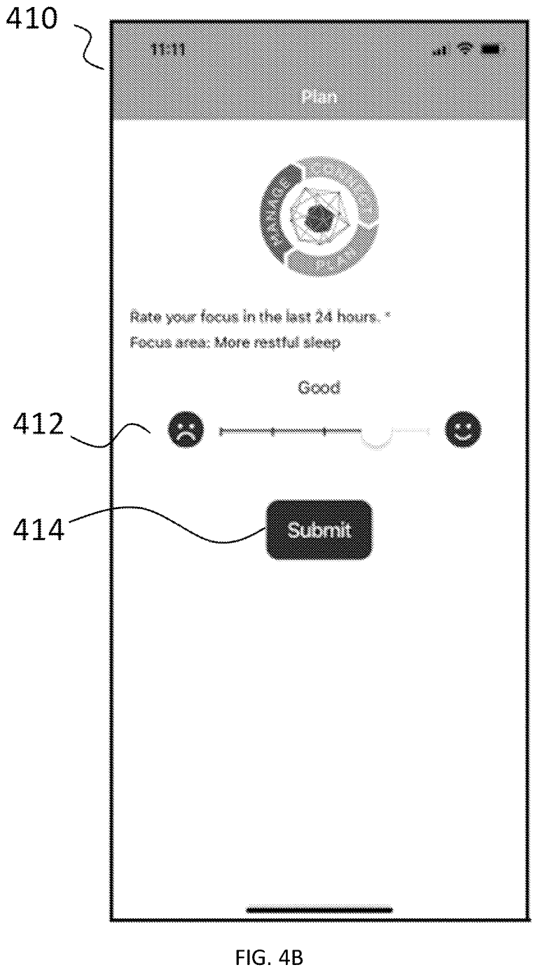

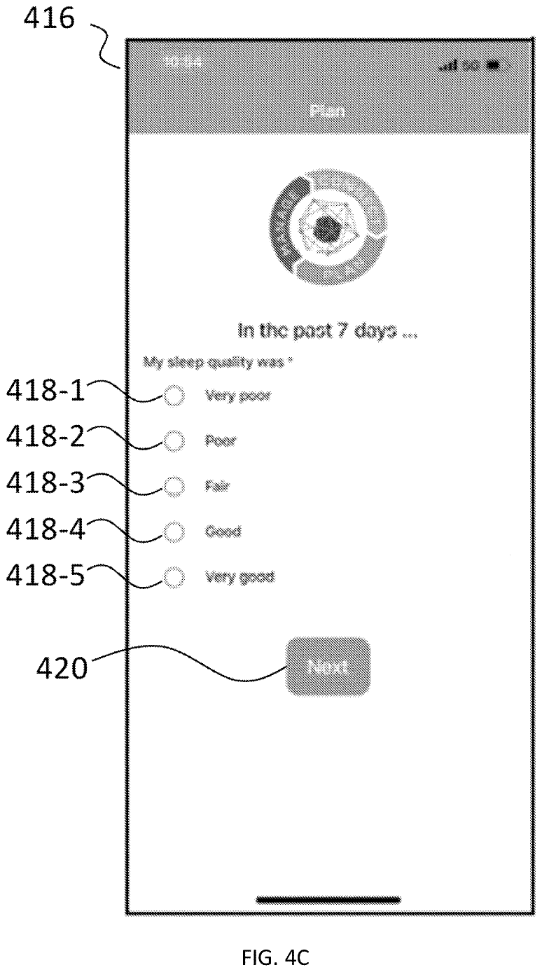

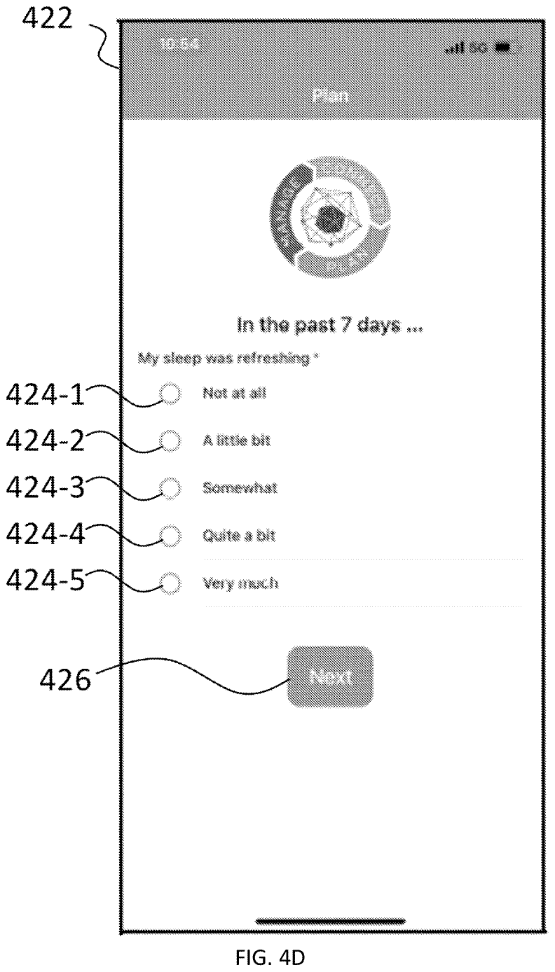

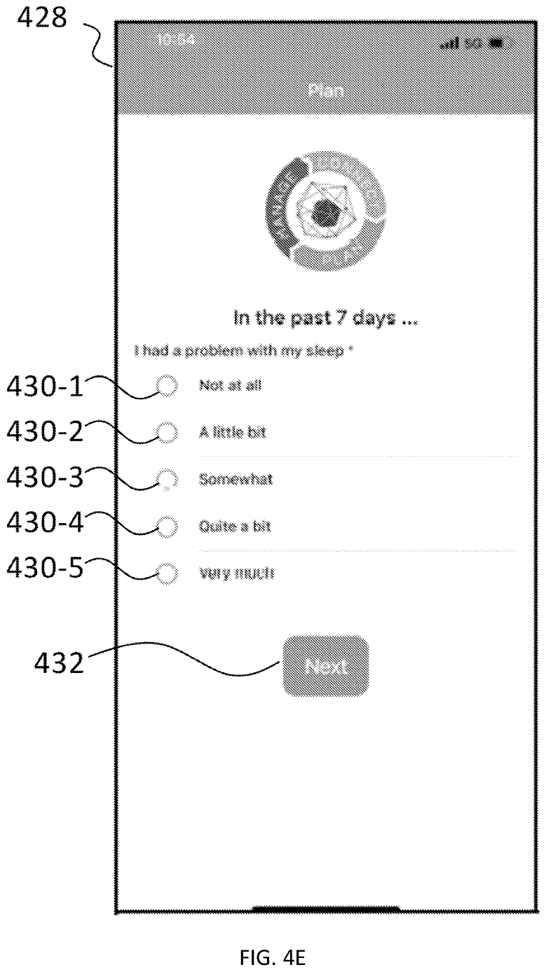

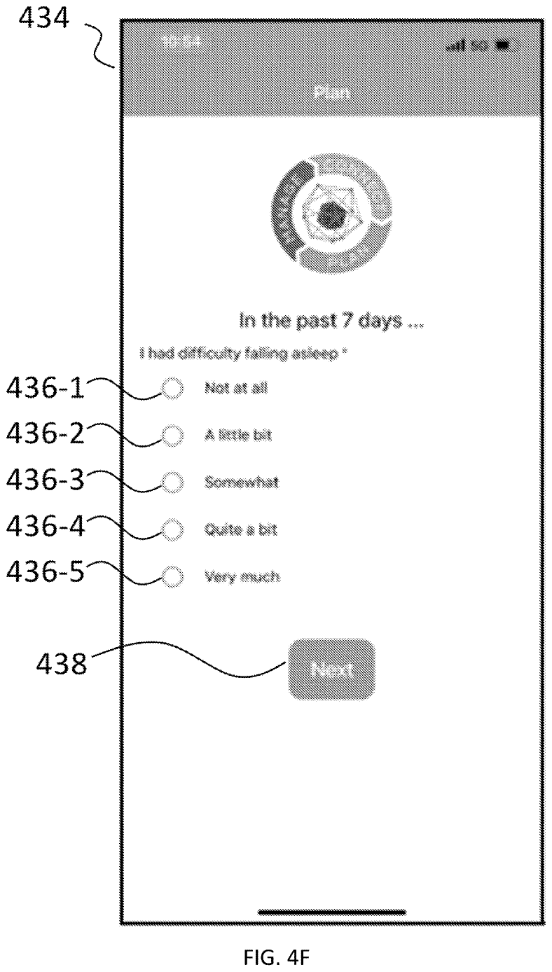

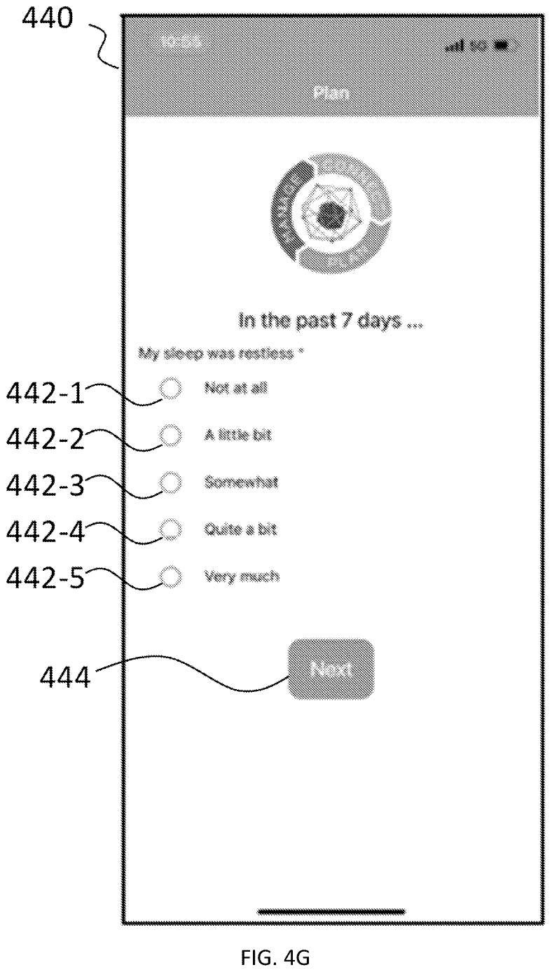

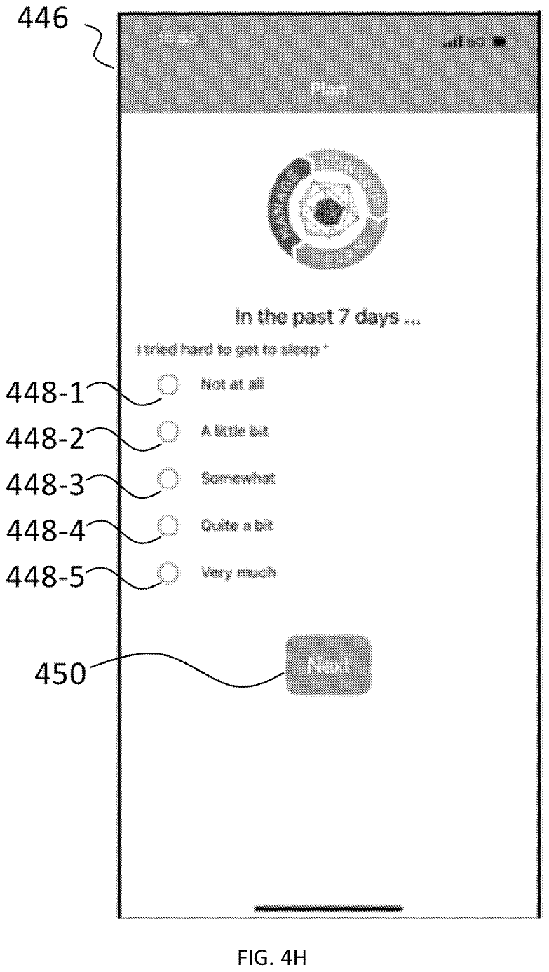

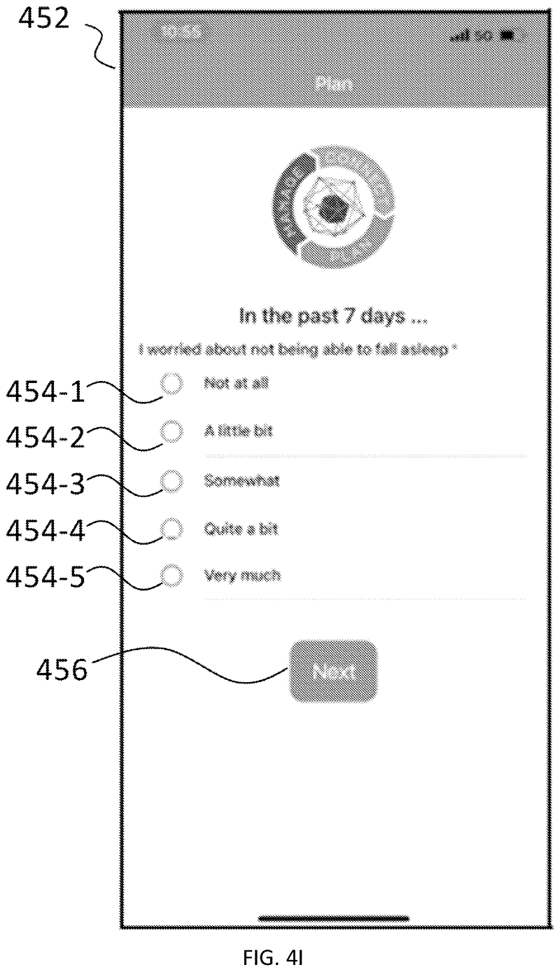

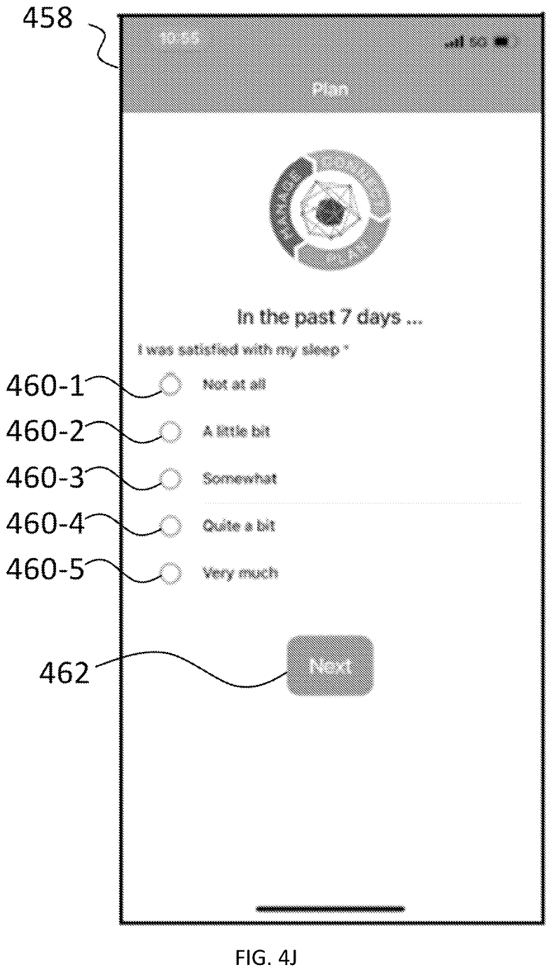

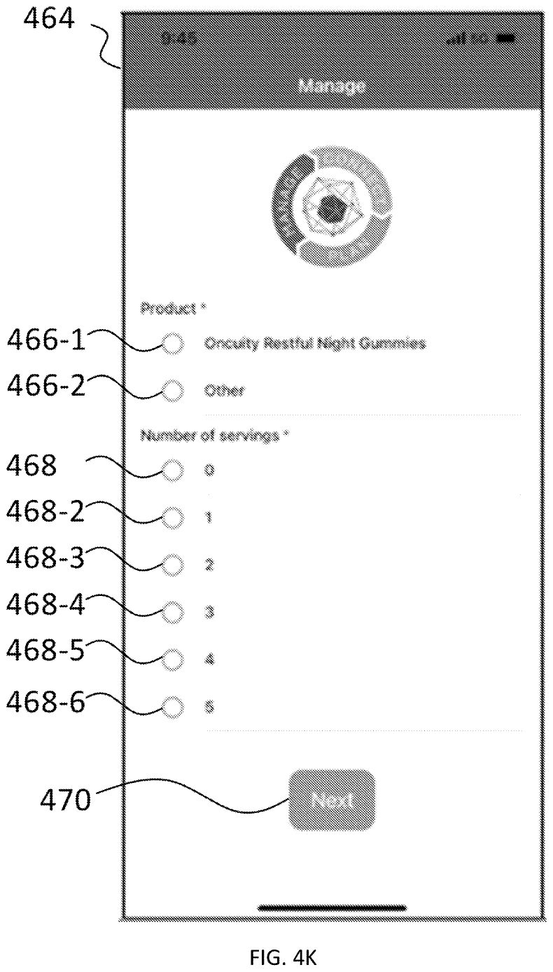

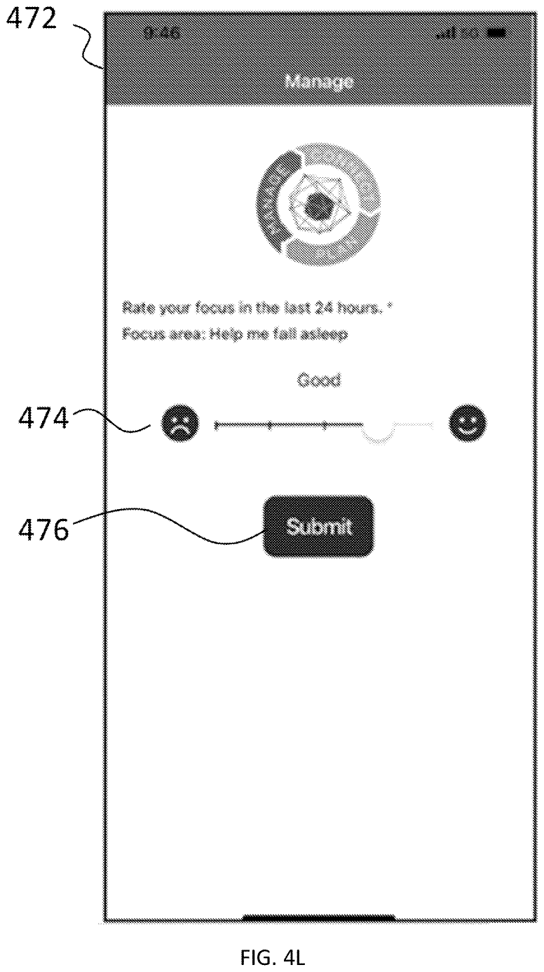

FIGS. 4A-4J illustrate examples of GUIs that may be provided to a subject during registration/enrollment at block 202 and/or after the CBT(s) have been prescribed and the subject is following a particular regimen at block 218. One of ordinary skill in the art will understand that all, some, or different GUIs may be presented to a subject, such as on a display 106 of a computing device 100, at various times.

The GUI 402 illustrated in FIG. 4A may present a question to a subject, such as “What is the main reason, your focus area, for using Oncuity Restful Night?” where “Oncuity Restful Night” is one example of a CBT in accordance with some embodiments. GUI 402 may also include a plurality of radio buttons 404-1, 404-2, . . . , 404-6 (collectively, “radio buttons 402”) that are configured to receive the subject input in the form of a selection of one of the predetermined answers to the presented question. Examples of the potential answers corresponding to a radio buttons 402 include, but are not limited to, “Help me fall asleep” 404-1, “Help me stay asleep” 404-2, “Improve sleep quality” 404-3, “More refreshing sleep” 404-4, “More restful sleep” 404-5, and “Other” 404-6. GUI 402 may also include an input field 406 in which the user may identify other reasons for having an interest in the CBT. In some embodiments, input field 406 may be deactivated unless and/or until the radio button 404-6 is selected. GUI 402 may also include a virtual button 408 labeled “Next” that, when selected by a user using an input device 132, may take the subject user to another GUI.

FIG. 4B illustrates another example of a GUI 410 that may be configured to receive feedback and/or other information from a user concerning CBT use in accordance with some embodiments. The GUI 410 may include a slider 412 that may enable a user to provide an answer to the presented request to “Rate your focus in the last 24 hours.” The subject user may move the slider in first and second directions (e.g., left/right or up/down) to provide a response to the request. A virtual button 414 may be labeled “Submit,” which submits the response when selected by the user. Upon selecting the virtual button 414, the data may be stored locally, such as in main memory 108 and/or secondary memory 110, and/or may be stored in a remote data store, such as data store 24. In some embodiments, the response provided by the subject user may correspond to a numeric or other value that may be stored in a profile associated with the subject user. The collected data, as described below, may be stored in a data store, such as data store 24, and analyzed by a processor, such as processor 22.

FIG. 4C illustrates another example of a GUI 416 in accordance with some embodiments. The GUI 416 may include one or more radio buttons 418-1, 418-2, . . . , 418-5 (collectively, “radio buttons 418”), which may correspond to an end of the partial sentence presented on the GUI, such as “My sleep quality was.” In some embodiments, the subject user may selected one of the radio buttons 418 corresponding to a description of the sleep, such as “Very poor” 418-1, “Poor” 418-2, “Fair” 418-3, “Good” 418-4, and “Very good” 418-5. GUI 416 may also include a virtual button 420 labeled “Next” that, when selected by a user using an input device 132, may take the subject user to another GUI. Upon selecting the virtual button 420, the data may be stored locally, such as in main memory 108 and/or secondary memory 110, and/or may be stored in a remote data store, such as data store 24. In some embodiments, the response provided by the subject user may correspond to a numeric or other value that may be stored in a profile associated with the subject user. The collected data may be stored in a data store, such as data store 24, and analyzed by a processor, such as processor 22 of hub network 20.

The example of a GUI 422 illustrated in FIG. 4D is shown as presenting the statement “My sleep was refreshing,” to which a subject user may respond using a radio button “Not at all” 424-1, “A little bit” 424-2, “Somewhat” 424-3, “Quite a bit” 424-4, and “Very much” 424-5. GUI 422 may also include a virtual button 426 labeled “Next” that, when selected by a user using an input device 132, may take the subject user to another GUI. Like with the other data collection GUIs described herein, the answers provided by a subject user, such as by selecting one of the radio buttons 424, may be stored in a profile for the user and analyzed by the one or more processors 22 of the hub network 20.

In the example illustrated in FIG. 4E, a GUI 428 may be provided presenting the statement “I had a problem with my sleep” with radio buttons corresponding to the answers “Not at all” 430-1, “A little bit” 430-2, “Somewhat” 430-3, “Quite a bit” 430-4, and “Very much” 430-5. A virtual button 432 labeled “Next” may be provided that causes the response provided by the user based on the selection of a radio button 430 to be recorded. The recorded response may be stored locally and/or remotely in a profile associated with the subject user.

Another example of a data collection GUI 434 is shown in FIG. 4F. As shown in FIG. 4F, the GUI 434 may present the statement “I had difficulty falling asleep” to which the subject user may respond, by using one of a plurality of radio buttons 436, “Not at all” 436-1, “A little bit” 436-2, “Somewhat” 436-3, “Quite a bit” 436-4, and “Very much” 436-5. Selection of the virtual button 438, which may be labeled “Next,” may cause the response input by the subject user (or a numerical or other value corresponding to the response) to be recorded. Here again, like with the other data collected by the app running on the computing device 100, the response data may be stored locally in a main memory 108 and/or a secondary 110 and/or may be stored in a remote memory, such as data store 24.

FIG. 4G illustrates another example of a GUI 440 that may be configured to receive feedback and/or other information from a user concerning CBT use in accordance with some embodiments. The GUI 440 illustrated in FIG. 4G may present the statement “My sleep was restless” to elicit one of a plurality of responses from the subject user via a radio button 442. Examples of such responses may include, but are not limited to, “Not at all” 442-1, “A little bit” 442-2, “Somewhat” 442-3, “Quite a bit” 442-4, and “Very much” 442-5. The subject user may provide a response, which may be submitted by pressing or otherwise selecting the virtual button 444 labeled “Next.” Submission of the responses may result in the responses being stored in a profile associated with the user, as described herein.

In the example illustrated in FIG. 4H, the GUI 446 may present the statement “I tried hard to get to sleep” to a user and offer one or more responses corresponding to a respective radio button 448. For example, the GUI may present the responsive statements “Not at all” corresponding to radio button 448-1, “A little bit” corresponding to the radio button 448-2, “Somewhat” corresponding to radio button 448-3, “Quite a bit” corresponding to radio button 448-4, and “Very much” corresponding to radio button 448-5. A subject user may select a radio button 448 corresponding to the most applicable statement and press the virtual button 450 labeled “Next” to store record the response.

In the example of a data collection GUI 452 is shown in FIG. 4I, the GUI presents the statement “I worried about not being able to fall asleep” and presents a number of selectable radio buttons 454 to the user. Each radio button 454 may correspond to a responsive statement, such as “Not at all” 454-1, “A little bit” 454-2, “Somewhat” 454-3, “Quite a bit” 454-4, and “Very much” 454-5. A subject user may select the appropriate radio button 454 and press or select the virtual button 456, which may be labeled “Next,” to have the answer recorded.

Another example of a data collection GUI is illustrated in FIG. 4J. The GUI 458 may include a statement, such as “I was satisfied with my sleep,” to which a plurality of responsive statements may be provided along with a corresponding radio button 460. For example, the GUI 458 may include a radio button 460-1 corresponding to the statement “Not at all,” a radio button 460-2 corresponding to the statement “A little bit,” a radio button 460-3 corresponding to the statement “Somewhat,” a radio button 460-4 corresponding to the statement “Quite a bit,” and a radio button 460-5 corresponding to the statement “Very much.” It should be understood that the manner in which information is elicited from a subject may change. For example, various types of questions may be presented and/or different responsive statements may be provided. In some embodiments, a sliding scale may be provided and/or a user may be asked to rate on a scale (e.g., 1-10). A virtual button 462 also may be provided, which may cause the entered response(s) to be stored once pressed or otherwise selected.

FIG. 4K illustrates another example of a GUI 464 that may be presented to a subject user to collect RWE and/or RWD to evaluate the safety and/or efficacy of one or more CBTs in accordance with some embodiments. In the example illustrated in FIG. 4K, the GUI 464 may be configured to acquire data concerning the type of CBT and/or dosing regimen. For example, GUI may include a first set of radio buttons 466 and a second set of radio buttons 468. Radio buttons 466-1, 466-2 may be associated with a product that the subject user is taking and/or prescribed, and radio buttons 468-1, 468-2, . . . , 468-6 may be associated with a specific dosage regimen, such as a number of gummies taken per day. It should be understood that the GUI 464 may have other configurations and/or layouts to collect data concerning the CBT regimen. In some embodiments, if the user selects the radio button 466-2 corresponding to the answer “Other” as to which product the subject is taking or has been prescribed, another GUI may be displayed (not shown) that may prompt the user to identify the other CBTs the user is taking and/or prescribed.

In the example GUI 472 illustrated in FIG. 4L, the GUI may be provided with a slider 474 so the user can rate the focus in the last 24 hours. When the user has moved the slider to a desired location, the virtual button 476, which may be labeled “Submit,” may be pressed or otherwise selected to record the user feedback. As described above, the collected data may be stored locally, such as in main memory 108 and/or secondary memory 110, and/or may be stored in a remote data store, such as data store 24. In some embodiments, the response provided by the subject user may correspond to a numeric or other value that may be stored in a profile associated with the subject user. For example, a subject may be assigned a unique identifier (e.g., a hash of a social security number, or other unique value that identifies a subject) and all of the records for the subject may be associated with the unique identifier. The collected data may be stored in a data store, such as data store 24, and analyzed by a processor, such as processor 22 of hub network 20.

In another example, the one or more sensors 70 may communicate sensor data to hub network 20. The sensor data communicated to hub network 20 may be indicative and/or effectiveness of one or more CBTs. For example, as discussed above, the sensor data transmitted from the one or more sensors 70 to hub network 20 may provide information concerning a subject's vital signs, sleep, and/or experienced pain. The hub network 20 may analyze the sensor data to assess the effectiveness and/or safety of one or more CBTs to identify whether the subject experienced any heart trouble or irregularity (e.g., arrhythmia, heart attack, spike in blood pressure) or had difficulty sleeping (e.g., frequent changing position and/or getting up) or breathing (e.g., low oxygen level and/or interrupted or irregular breathing).

The data collected by the app running on the computing device 100 and transmitted to hub network 20 may be used by subjects and other individuals, including researchers, medical professionals, CBT producers, actuaries, private and/or public insurance agencies, and government regulators, to list only a few possible examples of individuals that may have access to hub network 20. In some embodiments, the hub network 20, or the one or more processors 22 of hub network 20, may be configured to de-duplicate data collected from multiple sources for a single subject. For example, the hub network 20 may be configured to analyze data and map similar terms and/or fields of data into a single field or entry, such as by using machine learning, a look-up table, and/or other processing technique as will be understood by one of ordinary skill in the art. The collected raw data, such as data concerning mental health, physical health, wellness (e.g., sleep) and/or from one or more sensors 70, may be used to generate one or more GUIs to a subject user,

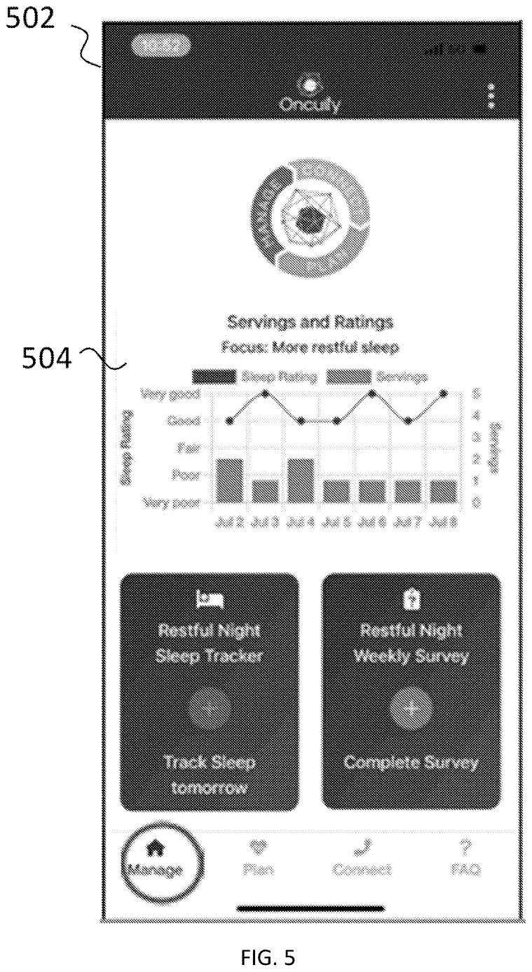

FIG. 5 illustrate one example of a GUI 502 that may be accessible via the app running on the computing device 100. The GUI 502 may include a graphical display 504, which may show a graphical representation of data collected over a period of time. For example, the graphical display 504 may include one or more representations of data indicating performance of the CBT. One of ordinary skill in the art will understand that a number of different metrics may be identified, calculation, and/or displayed, such as metrics representing the quality of slight on a nightly, weekly, and/or monthly basis; anxiousness and/or loneliness on a daily, weekly, and/or monthly basis; physical pain felt on an hourly, daily, weekly, and/or monthly basis; to list only a few possibilities. It should understood that these metrics may also be related to a CBT, including a type, dosage (e.g., time and quantity), and/or dosage related to other medications, for example.

As noted above, the collected and/or observed real-world data may be collected and analyzed by hub network 20. The analysis performed by hub network 20 may be used to develop and/or adjust a CBT for one or more subject users. Additionally or alternatively, the collected data may be accessed and/or used by one or more public and/or private insurance provider networks 50, which may analyze the collected data to assess whether the one or more CBTs may be a covered expense. The collected data may be also be used to develop one or more new payment and/or service delivery models, such as through CMMI.

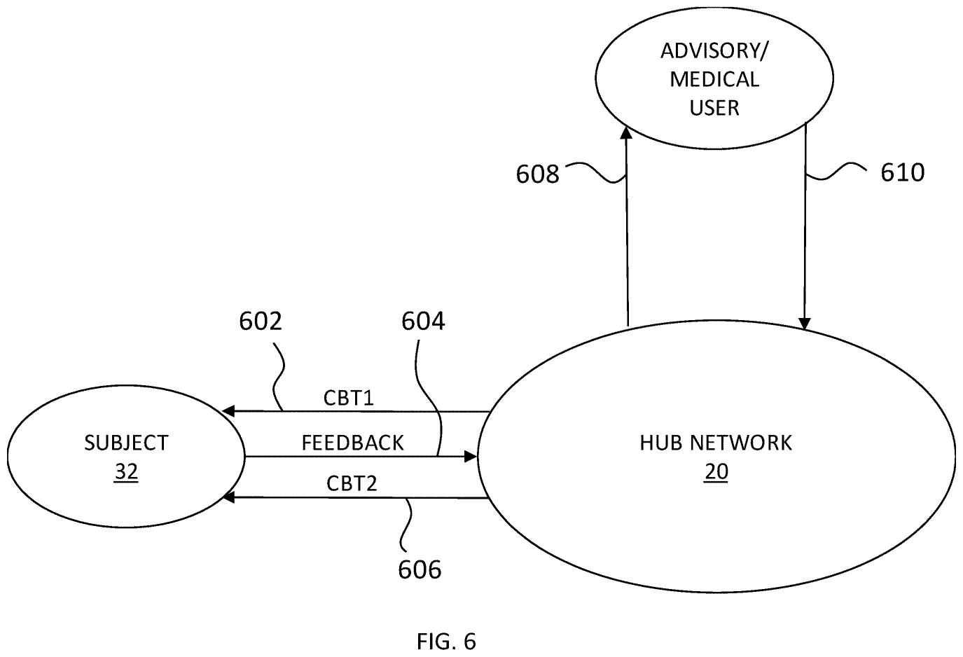

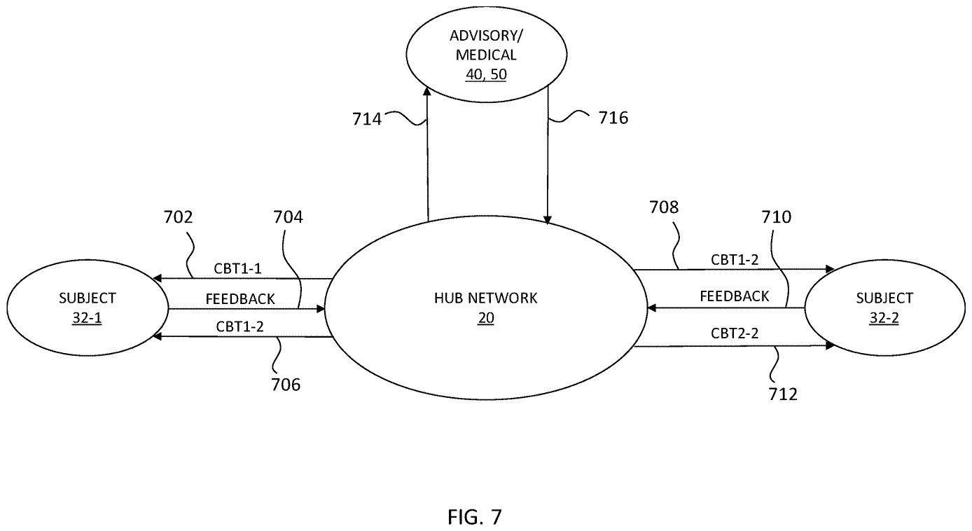

FIGS. 6 and 7 illustrate example flows of how the hub network 20 may be configured to develop and/or adjust one or more CBTs in accordance with some embodiments. In the example illustrated in FIG. 6, a CBT may be prescribed by the hub network 20 and then modified based on feedback from a subject 32. It should be understood that the subject 32 may be a user of a computing device, such as computing device 100-1, 100-2 illustrated in FIG. 1B. Further, the initial prescription and/or suggestion of a CBT may be provided by the hub network 20 in response to receiving the prescription and/or suggestion from a medical advisor or professional who may enter the prescription or recommendation using one or more computing devices, such as one or more of computing devices 100-3, 100-4, 100-5 of one or more of researcher/advisory/producer network(s) 40 and/or computing devices 100-6, 100-7, 100-8 of provider network(s) 50.

As shown in FIG. 6, an initial CBT, identified as CBT1, may be prescribed or otherwise recommended to subject 32 represented by arrow 602. In some embodiments, the initial CBT1 is provided from hub network 20 and may be based on data collected during a registration and/or enrollment process, such as the enrollment/registration process described above with respect to block 202 in FIG. 2. CBT1 may include one or more active ingredients having a first formulation, such as certain percentage weights and/or ratios of each active ingredients, along with a specific dosing regimen (e.g., frequency of administration, time of day of administration, etc.). For example, CBT1 may be prescribed to be used at a certain time (e.g., morning, afternoon, evening) and/or at a certain interval (e.g., a certain time before sleep, periodically throughout the day, etc.).

As noted above, the CBT that is prescribed or recommended may be one of a plurality of different CBTs, such as CBTs having different form factors (e.g., tincture, gummy, lozenge, vape) with different active ingredients (e.g., cannabidiol (CBD), Cannabinol (CBN), melatonin, theanine, tea extract, etc.) and/or flavors (e.g., cherry, lemon, tangerine, blueberry, etc.).

In one embodiment, the CBT is in a tincture (see, e.g., Pertwee, British Journal of Pharmacology (2006) 147, S163-S171). In another embodiment, the CBT is a product to be administered orally, such as a gummy (which may be formulated with gelatin or pectin), lozenge (which may be formulated as a medicated candy) or vape cartridge (which may be a glass cartridge containing the CBT in the form of an oil), the formulations of which are well known to one of skill in the art. In another embodiment, the CBT may be topically administered, for example, in the form of an oil, lotion, stick or transdermal patch (e.g., a sticker, optionally with lidocaine, infused with CBT). Methods of making an oil, lotion, stick or transdermal patch are well known to one of skill in the art.

In an embodiment, wherein the CBT is formulated as a tincture, the tincture of U.S. Patent Publication No. 2022/0153717, the disclosure of which is incorporated by reference, may be used. A cannabinoid and solvent mixture may be heated, evaporated and removing and collecting the cannabinoid crude oil.

In an embodiment, wherein the CBT is formulated as a gummy candy, the gummy of U.S. Patent Publication No. 2020/0037638, the disclosure of which is incorporated by reference, may be used. A sample amount of gummy base ingredient per batch size is provided below:

### Amount of Gummy Base Ingredient Per Batch Size

| | 1X | | 2X | | 10X | | 20X | |
|---|---|---|---|---|---|---|---|---|
| Gelatin | 65 | g | 130 | g | 650 | g | 1300 | g |
| (280 | | | | | | | | |
| bloom) | | | | | | | | |
| Water | 165 | g | 330 | g | 1650 | g | 3300 | g |
| Sugar | 225 | g | 450 | g | 2250 | g | 4500 | g |
| Corn | 245 | g | 490 | g | 2450 | g | 4900 | g |
| Syrup | | | | | | | | |
| Xylitol | 26 | g | 52 | g | 260 | g | 520 | g |
| Citric | 45 | g | 90 | g | 450 | g | 900 | g |
| Acid | | | | | | | | |
| Flavor | 15 | g | 30 | g | 150 | g | 300 | g |
| Color | 1 | Drop | 2 | Drops | 10 | Drops | 20 | Drops |

1. Ingredients may be scaled to a desired size. Gelatin and water may be combined and mixed well. The mixture may immediately begin to bloom.

2. Sugar, xylitol and corn syrup may be combined in a pot and heated on a stove until it reaches 250° F.

3. Bloomed gelatin may be added to the sugar mixture in semi-small chunks and mixed well with a spatula until approximately all gelatin melts. Gelatin mixture may be weighed and amount of cannabinoid formulation required for desired dose may be calculated.

4. Color, flavor, cannabinoid formulation, and citric acid may be added to the gelatin mixture. The cannabinoid formulation may consist of cannabinoid extract dissolved in MCT (total percent between 10-80 w/v) and polysorbate 80 (total percent between 10-90 w/v). The ingredients may be mixed well with a mixer and poured into a funnel. Foam may be allowed to come to the top (5 minutes) before pouring.

5. The mixture may be poured into square pans sprayed with a non-stick spray. Foam may not be allowed to pour into pans. The funnel may topped off as needed with the remaining gummy mixture.

6. Trays may be transferred to a rolling rack and allowed to set up slightly before moving to a refrigerator.

7. Gummies may be cut into cubes. Each gummy cube typically contains a cannabinoid dose ranging from 1-10 mg.

In an embodiment, wherein the CBT is formulated as or includes a lozenge, the lozenge of U.S. Pat. No. 11,241,413, the disclosure of which is incorporated by reference, may be used. The CBT may be mixed with an alcohol, such as a sugar alcohol and formulated with one or more solid particles such as but not limited to silica, microcrystalline cellulose, cellulose, silicified microcrystalline cellulose, clay, talc, starch, pregelatinized starch, calcium carbonate, dicalcium phosphate, magnesium carbonate, magnesium-alumino-metasilicates, hyper porous silica and mixtures thereof.

In an embodiment, wherein the CBT is formulated as or includes a vape, the vape of U.S. Patent Publication No. 2020/0297025, the disclosure of which is incorporated by reference, may be adapted for such use. A vape oil may be manufactured by a) selecting a cannabinoid having a viscosity at room temperature which is above the viscosity at room temperature suitable for use in the vape device and having a flash point above the vaporization temperature; b) selecting an additive having a viscosity below the vape oil viscosity and having a flash point below the vaporization temperature, the additive operating to lower the viscosity of the cannabinoid source and operating to lower a flash point of a mixture of the cannabinoid source and the additive; c) determining a concentration of additive that (i) will reduce the viscosity of the cannabinoid source sufficiently low for the mixture to be suitable for use in the vape device and (ii) while avoiding reducing the flash point of the mixture below the vaporization temperature; and d) mixing the cannabinoid source of a) and the additive of b) on the basis of the concentration determined in c) to obtain the vape oil.

In an embodiment, wherein the CBT is formulated as a transdermal, the patch of any of U.S. Patent Publication No. 2019/0247453, or U.S. Patent Publication No. 2021/0228497, or U.S. Patent Publication No. 2019/0110981, the disclosure of each of which is incorporated by reference, may be adapted for such use. Transdermal delivery can also be by way of a CBT ointment, or CBT spot-on formulation. It is understood that a spot-on formulation is as in the veterinary health field, namely a concentrated solution that is applied as a spot, and with solvent or co-solvent evaporation there is a concentration gradient between the outer skin surface and the inner skin surface that drives the active, in this case CBT (e.g., CBN, CBD, and optionally melatonin and/or L-theanine), to penetrate the skin. Thus, disclosures such as those of WO 2007018659 and U.S. Pat. Nos. 10,898,467, 6,426,333, 6,482,425, and 6,395,765, and the documents cited in these publications, all hereby incorporated herein by reference, can be modified whereby the anti-parasitic (e.g., phenylpyrazole, macrocyclic lactone endectocidal parasiticide, insect growth hormone mimetic) is replaced by CBT (e.g., CBN, CBD, and optionally melatonin and/or L-theanine (e.g., 10-30% by weight CBT (e.g., combination of CBN, CBD, melatonin and L-theanine as discussed herein for gummy in proportions as in gummy) in an organic solvent such as N-methylpyrrolidone. For instance, a single topical dose of a spot-on can include about 20 mg CBD, or Full Spectrum Activated CBD and/or other cannabinoid Extract(s) (e.g., mixtures of CBD & CBN and/or CBD, CBC and CBG, e.g., in proportions as provided in other form factors disclosed herein, e.g., in proportions as provided for herein for other formulations, such as for tincture or gummy form discussed herein and/or tabulated herein, starting from the total cannabinoids being 20 mg in an embodiment of a transdermal formulation).

The CBT may comprise a plurality one or more cannabinoids, ideally about 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 250 mg, 300 mg, 350 mg, 400 mg, 450 mg, 500 mg, 750 mg, 1000 mg, 1500 mg, 2000 mg, 2500 mg, or 3000 mg of a cannabinoid. If the cannabinoid is in a liquid, the oral solution may be about 10 mg/mL, 20 mg/mL, 30 mg/mL, 40 mg/mL, 50 mg/mL, 60 mg/mL, 70 mg/mL, 80 mg/mL, 90 mg/mL or 100 mg/mL of a cannabinoid. In an advantageous embodiment, there is about 50 mg or 50 mg/mL of cannabinoid in the CBT. In some embodiment, the dose of CBT may be about 1 mg/kg, about 2 mg/kg, about 3 mg/kg and may be up to about 10 mg/kg/day, about 15 mg/kg/day, about 20 mg/kg/day or about 25 mg/kg/day.

If the cannabinoid is admixed into a solid composition such as in a stick or a balm, the total amount of cannabinoid in the composition may be about 0.1%, 0.2%, 0.3%, 0.4%, 0.5%, 0.6%, 0.7% 0.8%, 0.9%, 1%, 2%, 3%, 4%, 5%, 6%, 7%, 8%, 9%, 10%, 15%, 20%, 25%, 30%, 35%, 40%, 45%, 50%, 55%, or 60% by weight of the total composition. In an advantageous embodiment, the CBT contains about 3% by weight of the total composition of cannabinoid. In some embodiments, the total mass of the solid composition, which includes the active cannabinoids and other non-active ingredients, can be about 5 g, 10 g, 15 g, 20 g, 25 g, 30 g, 35 g, 40 g, 45 g, 50 g, 55 g, 60 g, 65 g, 70 g, 75 g, 80 g, 85 g, 90 g, 95 g, 100 g, 150 g, 200 g, 200 g, 250 g, 300 g, 350 g, or 400 g.

Cannabinoids and their abbreviations are presented in the table below:

| | | |
|---|---|---|
| CBD | Cannabidiol | 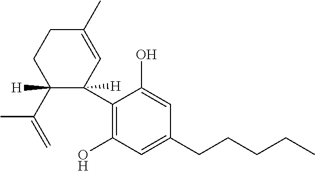 [SMILES: C=C(C)[C@@H]1CCC(C)=C[C@H]1c1c(O)cc(CCCCC)cc1O] |
| CBDA | Cannabidiolic acid | 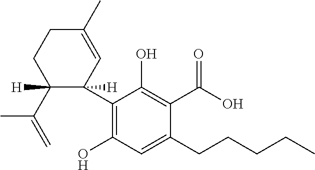 [SMILES: C=C(C)[C@@H]1CCC(C)=C[C@H]1c1c(O)cc(CCCCC)c(C(=O)O)c1O] |
| CBDV | Cannabidivarin | 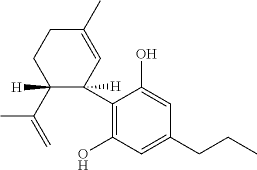 [SMILES: C=C(C)[C@@H]1CCC(C)=C[C@H]1c1c(O)cc(CCC)cc1O] |
| CBDVA | Cannabidivarinic acid | 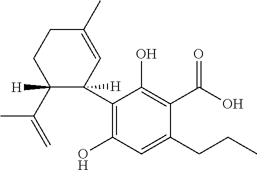 [SMILES: C=C(C)[C@@H]1CCC(C)=C[C@H]1c1c(O)cc(CCC)c(C(=O)O)c1O] |
| THC | Tetrahydrocannabinol | 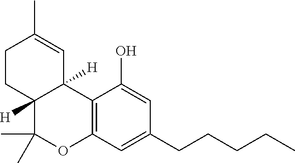 [SMILES: CCCCCc1cc(O)c2c(c1)OC(C)(C)[C@@H]1CCC(C)=C[C@@H]21] |

The table above is not exhaustive and merely details the cannabinoids, which are identified in the present application for reference. So far, over 60 different cannabinoids have been identified and these cannabinoids can be split into different groups as follows: phytocannabinoids; endocannabinoids; and synthetic cannabinoids (which may be novel cannabinoids or synthetically produced phytocannabinoids or endocannabinoids).

“Phytocannabinoids” are cannabinoids that originate from nature and can be found in the cannabis plant. The phytocannabinoids can be isolated from plants to produce a highly purified extract or can be reproduced synthetically.

“Highly purified cannabinoids” are defined as cannabinoids that have been extracted from the cannabis plant and purified to the extent that other cannabinoids and non-cannabinoid components that are co-extracted with the cannabinoids have been removed, such that the highly purified cannabinoid is greater than or equal to 95% (w/w) pure.

“Synthetic cannabinoids” are compounds that have a cannabinoid or cannabinoid-like structure and are manufactured using chemical means rather than by the plant.

Phytocannabinoids can be obtained as either the neutral (decarboxylated form) or the carboxylic acid form depending on the method used to extract the cannabinoids. For example, it is known that heating the carboxylic acid form will cause most of the carboxylic acid form to decarboxylate into the neutral form.

The cannabinoid advantageously may be CBD, CBN, cannabichromene (“CBC”) and/or cannabigerol (CBG) or any combination thereof. In a particularly advantageous embodiment, there is about 40 mg, 45 mg or 50 mg pf CBD. In an embodiment, where there is less than 50 mg of CBD, there is advantageously an amount of CBC and or CBG so there is about 50 mg of cannabinoid in the CBT. For example, if there is 40 mg of CBD in the CBT, there may be about 10 mg of CBN or about 5 mg of CBD and 5 mg of CBG.

CBD may be a highly purified as described in U.S. Pat. Nos. 10,849,860; 11,065,209; and 10,966,939 the disclosures of which is incorporated by reference in its entirety.

In another embodiment, the CBT may further comprise additional active ingredients, such as but not limited to melatonin and theanine. Melatonin has been used for short-term treatment of trouble sleeping (insomnia) due to sleep cycle disorders and time changes. A dose of about 0.1 mg, 0.2 mg, 0.3 mg, 0.4 mg, 0.5 mg., 0.6 mg, 0.7 mg, 0.8 mg, 0.9 mg, 1 mg, 2 mg, 3 mg, 4 mg, 5 mg, 6 mg, 7 mg, 8 mg, 9 mg or 10 mg of melatonin is contemplated. In an advantage embodiment, there may be about 1 mg to about 3 mg of melatonin. In a particularly advantageous embodiment, the dosage of melatonin may be about 1 mg.

Theanine is an amino acid analogue of the proteinogenic amino acids L-glutamate and L-glutamine which yay help reduce stress and anxiety acutely in people with acute stress. A dose of about 10 mg, 20 mg, 30 mg, 40 mg, 50 mg, 60 mg, 70 mg, 80 mg, 90 mg, 100 mg, 200 mg, 300 mg, 400 mg, 500 mg, 600 mg, 700 mg, 800 mg, 900 mg or 1000 mg of theanine is contemplated. In an advantageous embodiment, about 50 mg to about 250 mg of theanine is contemplated. In a particularly advantageous embodiment, the dosage of theanine may be about 50 mg.

In another embodiment, a tea extract is also contemplated as an active ingredient. In an advantageous embodiment, the tea extract may comprise one or more catechins. In a particularly advantageous embodiment, the catechin may be epigallocatechin gallate (EGCG). A dose of about 0.1 mg, 0.2 mg, 0.3 mg, 0.4 mg, 0.5 mg., 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 200 mg, 300 mg, 400 mg or 500 mg of EGCG is contemplated. In an advantageous embodiment, about 1 mg to about 100 mg of EGCG is contemplated. In a particularly advantageous embodiment, the dosage of EGCG may be about 2.5 mg.

In another embodiment, a local anesthetic is also contemplated as an active ingredient, which can be simultaneously delivered along with the cannabinoid such as, for example, in a transdermal patch. In some embodiments, the local anesthetic may include benzocaine, procaine, chloroprocaine, prilocaine, and lidocaine. In some embodiments, the transdermal patch may comprise about 1%, about 2%, about 3%, about 4%, about 5%, about 6%, about 7%, about 8%, about 9%, about 10%, about 15%, about 20%, about 25%, about 30%, about 35%, or about 40% by weight of the local anesthetic based on the total weight of active ingredients.

A plurality of flavors are contemplated for the CBTs, in particular the oral CBTs. Advantageously, the flavor includes a fruit flavor. The fruit flavor may include, but is not limited to, apple, apricot, banana, blackberry, blueberry, cantaloupe, cherry, coconut, cranberry, date, grape, grapefruit, guava, honeydew, kiwi, kumquat, lemon, lime, lychee, mango, nectarine, orange, papaya, passion fruit, peach, pear, persimmon, pineapple, plum, pomegranate, pumpkin, quince, raspberry, rhubarb, strawberry, or any combination thereof. The flavor may also include a floral or an herbal flavor. The floral flavor may include, but is not limited to jasmine, violet, elderflower, rose, lavender, and hibiscus. The herbal flavor may include, but is not limited to mint, peppermint, hemp, chamomile, rosemary, ginger, turmeric, and sage. Additional sweeteners (such as sucrose or sucralose) are also contemplated to improve palatability.

Additional components and/or excipients are also contemplated such as for example, an oil (such as sesame oil) to solubilize the CBTs, an alcohol (such as ethanol) to solubilize a sweetener and/or flavoring.

Miscellaneous ingredients are also contemplated, including natural ingredients for color and flavor, oils, syrups, waxes and acids, the addition of which is within the purview of one of skill in the art.

The CBT may include one or more essential oils to stimulate the olfactory system for the improvement of psychological and physical well-being. Essential oils also emit a variety of aromas that may also induce a sense of calm and relaxation. Examples of essential oils that can be used include, but are not limited to peppermint oil, rosemary oil, tea tree oil, lemongrass oil, elemi oil, lavender oil, basil oil, eucalyptus oil, geranium oil, juniper oil, palmrose oil, passion fruit oil, patchouli oil, rosewood oil, sage oil, thyme oil, and ylang ylang oil, and combinations thereof.

Other ingredients may also include one or more terpenes to enhance the effects of the CBT. Terpenes as used in a CBT may include, but are not limited to myrcene, linalool, limonene, pinene, and caryophyllene. Terpenes have a variety of applications in CBT and may assist in providing aromatherapy via stimulation of the olfactory system, as well as assist in relieving stress and anxiety, improving mood, and improving sleep. Terpenes may also increase the absorption rate of cannabinoids to enhance their effect. Other benefits that terpenes may provide include, but are not limited to analgesic, antibacterial, and anti-inflammatory properties.

In some embodiments, a CBT may include a gummy having a serving size comprising about 2.4 g to about 3.0 g, e.g., about 2.7 g, which contains about 0.5 mg to about 1.5 mg, e.g., about 1 mg of melatonin, about 40 mg to about 60 mg, e.g., about 50 mg of L-theanine, about 42 mg to about 62 mg, e.g., and about 52 mg of hemp extract, along with other non-active ingredients, wherein the total phytocannabinoids thereof are about 40 mg to about 60 mg, e.g., about 50 mg, and wherein the total phytocannabinoids comprise about 30 mg to about 50 mg of cannabidiol (CBD) and about 5 mg to about 20 mg, e.g., about 10 mg cannabinol (CBN), with other non-active ingredients comprising a sweetener such as tapioca syrup, e.g., organic tapioca syrup, a sugar, such as cane sugar, e.g., organic cane sugar, water, soluble polysaccharide, such as pectin, an acidifier (that may also add flavor and thus also serve as a flavor agent and/or that may also act as a chelating agent and/or that may act as an emulsifier and/or that may act as a preservative), e.g., citric acid, flavor, e.g., a cherry flavor, medium chain triglyceride oil (MCT oil, e.g., based on caproic acid (C6) and/or caprylic acid (C8) and/or capric acid (C10) and/or lauric acid (C12), e.g., the MCT oil can be a mixture of oils based on any one or more of the foregoing acids) that may be useful for solubilizing or dissolving one or more active ingredients (e.g., the CBD and/or CBN can be admixed and dissolved into the MCT) as well as providing a profile for the actives to more rapidly take effect by more rapidly passing from the gut to the bloodstream, a salt of the acidifier, e.g., for pH balance, e.g., sodium citrate, a coloring agent, e.g., elderberry juice, and a thickener, e.g., a wax, such as carnauba wax. Accordingly, such an exemplary gummy can be about 2.7 g total weight and contain about 1 mg melatonin, about 50 mg L-theanine, about 52 mg of hemp extract having about 50 mg phytocannabinoids comprising about 40 mg cannabidiol (CBD) and about 10 mg cannabinol (CBN). Such an exemplary gummy can be about 10 calories per gummy and provide about 2 g of carbohydrate or total sugars. Such an exemplary gummy can be a sleep aid. The dosing of such an exemplary gummy can be one (1) gummy to be taken at about 30 minutes prior to bedtime, and to be increased to two (2) gummies to be taken at about 30 minutes prior to bedtime if one gummy does not provide the desired effect of aiding in the subject going to sleep. It should be understood that other doses and dosing regimens may be used, including taking one or more gummies on an order of minutes to hours before bed.

In some embodiments, the gummy may be used as an aid for relief from anxiety and stress. In some embodiments, the gummy may be used to uplift one's mood and provide an energy boost or a state of euphoria. In some embodiments, the gummy may be used as an aid for pain relief. The gummy can be taken on demand to provide the associated benefits.

In some embodiments, a CBT may include a tincture having a serving size between about 0.25 mL and about 1 mL, wherein the tincture contains about 0.5 mg/ml to about 1.5 mg/mL, e.g., about 1 mg/mL of melatonin, about 40 mg/mL to about 60 mg/mL, e.g., about 50 mg/mL of L-theanine, along with other non-active ingredients, wherein the total phytocannabinoids thereof are about 40 mg/mL to about 60 mg/mL, e.g., about 50 mg/mL, and wherein the total phytocannabinoids comprise about 30 mg/mL to about 50 mg/mL of cannabidiol (CBD) and about 5 mg/mL to about 20 mg/mL, e.g., about 10 mg/mL cannabinol (CBN), with other non-active ingredients comprising a flavoring agent, medium chain triglyceride oil (MCT oil, e.g., based on caproic acid (C6) and/or caprylic acid (C8) and/or capric acid (C10) and/or lauric acid (C12), e.g., the MCT oil can be a mixture of oils based on any one or more of the foregoing acids) that may be useful for solubilizing or dissolving one or more active ingredients (e.g., the CBD and/or CBN can be admixed and dissolved into the MCT) as well as providing a profile for the actives to more rapidly take effect by more rapidly passing from the gut to the bloodstream, and one or more terpenes that stimulate the olfactory system via their aromatic scent, may provide a profile for the actives to more rapidly take effect by increasing the absorption rate of the active ingredients, and may also provide analgesic, antibacterial, and anti-inflammatory effects. Accordingly, such an exemplary tincture can contain about 1 mg/mL melatonin, about 50 mg/mL L-theanine, about 50 mg/mL phytocannabinoids comprising about 40 mg/mL cannabidiol (CBD) and about 10 mg/mL cannabinol (CBN). Such an exemplary tincture can be a sleep aid. The dosing of such an exemplary tincture can be 1 mL of the tincture placed under the tongue approximately 30 minutes prior to bedtime. It should be understood that other doses and dosing regimens may be used, including increasing or decreasing the tincture taken on an order of minutes to hours before bed.

In some embodiments, the tincture may be used as an aid for relief from anxiety and stress. In some embodiments, the tincture may be used to uplift one's mood and provide an energy boost or a state of euphoria. The tincture may be taken on demand to provide the associated benefits.

In some embodiments, a CBT may include a stick that may be applied as needed to a site that may be suffering from, for example but not including, pain, soreness, achiness, and cramping. The total phytocannabinoids thereof are about 500 mg to about 2500 mg, and wherein the total phytocannabinoids comprise about 400 to 2400 mg of cannabidiol (CBD), about 5 to about 50 mg of cannabichromene (CBC), and about 5 to about 50 mg of cannabigerol (CBG) with other non-active ingredients can comprise coconut oil, kester wax, sunflower wax, organic cocoa butter, jojoba oil, rice bran oil, an essential oil blend, rosemary extract, and menthol. The stick can In some embodiments, the stick contains about 3% by weight of CBD, about 0.05% by weight of CBG, and about 0.01% by weight of CBC. The stick may be in the form of a solid stick, a balm, a gel, and may be applied to any affected part of the body as needed such as the back, neck, hip, shoulders, elbows, joints, or muscles.

In some embodiments, a CBT may include a transdermal patch that may be applied as needed to relieve pain. The total phytocannabinoids thereof are about 10 mg to about 100 mg, and the phytocannabinoids present may include, but are not limited to CBD, CBN, and CBG, or combinations thereof. In some embodiments, the transdermal patch contains about 10 mg, about 20 mg, about 30 mg, about 40 mg, about 50 mg, or about 50 mg of CBD. In some embodiments, the transdermal patch may comprise other active ingredients to assist in relieving pain such as a local anesthetic. In some embodiments, the local anesthetic is lidocaine. The patch can be any size or shape. In some embodiments, the transdermal patch is a square. In some embodiments, a square transdermal patch can have a size from about 1″×1″ to about 4″×4″.

The transdermal patch can be applied to a venous area of the skin and is worn for approximately 8 to 24 hours. The onset of the benefits of the patch takes approximately 30 to 45 minutes following application of the transdermal patch.

The following tables set forth additional examples of CBTs that may be prescribed and/or recommended, but it should be understood by one of ordinary skill in the art that other CBTs may be prescribed and/or recommended.

CBT_{A}

| | |
|---|---|
| Form factor: | Tincture |
| Flavor: | Cherry |
| Active | 40 mg/mL CBD |
| Ingredient(s): | 10 mg/mL CBN |
| | 1 mg/mL Melatonin |
| Other | MCT oil; cherry flavor; terpene blend (Beta- |
| Ingredient(s): | caryophyllene, D-Limonene, Linalool, |
| | Nerolidol, Myrcene) |

CBT_{B}

| | |
|---|---|
| Form factor: | Gummy |
| Flavor: | Cherry |
| Active | 40 mg CBD |
| Ingredient(s): | 10 mg CBN |
| | 1 mg Melatonin |
| | 50 mg L-Theanine |
| Other | Organic tapioca syrup; organic cane sugar; |
| Ingredient(s): | water; pectin; citric acid; natural cherry |
| | flavor; MCT oil; sodium citrate; elderberry |
| | juice (color); carnauba wax |

CBT_{C}

| | |
|---|---|
| Form factor: | Tincture |
| Flavor: | Lemon lavender |
| Active | 45 mg/mL CBD |
| Ingredient(s): | 5 mg/mL CBN |
| | 1 mg/mL Melatonin |
| | 50 mg/mL L-Theanine |
| Other | Organic tapioca syrup; organic cane sugar; |
| Ingredient(s): | water; pectin; citric acid; lemon oil; sodium |
| | citrate; turmeric (color); natural terpene blend |
| | (D-Limonene, Linalool); carnauba wax |

CBT_{D}

| | |
|---|---|
| Form factor: | Tincture |
| Flavor: | Lemon lavender |
| Active | 45 mg/mL CBD |
| Ingredient(s): | 5 mg/mL CBN |
| Other | MCT oil; pure lemon oil; terpene blend (D- |
| Ingredient(s): | Limonene, Linalool) |

CBT_{E}

| | |
|---|---|
| Form factor: | Gummy |
| Flavor: | Lemon lavender |
| Active | 45 mg CBD |
| Ingredient(s): | 5 mg CBN |
| | 50 mg L-Theanine |
| Other | Organic tapioca syrup; organic cane sugar; |
| Ingredient(s): | water; pectin; citric acid; lemon oil; sodium |
| | citrate; turmeric (color); natural terpene blend |
| | (D-limonene, Linalool); carnauba wax |

CBT_{F}

| | |
|---|---|
| Form factor: | Tincture |
| Flavor: | Tangerine |
| Active | 40 mg/mL CBD |
| Ingredient(s): | 5 mg/mL CBC |
| | 5 mg/mL CBG |
| Other | MCT oil; pure orange oil; pure tangerine oil; |
| Ingredient(s): | terpene blend (D-Limonene, Alpha-Pinene, |
| | Beta-Pinene, Beta-Caryophyllene) |

CBT_{G}

| | |
|---|---|
| Form factor: | Gummy |
| Flavor: | Tangerine |
| Active | 40 mg CBD |
| Ingredient(s): | 5 mg CBC |
| | 5 mg CBG |
| | 2.5 mg green tea extract (EGCG) |
| Other | Organic tapioca syrup; organic cane sugar; |
| Ingredient(s): | water; pectin; citric acid; pure tangerine oil; |
| | sodium citrate; beta-carotene (color); ascorbic |
| | acid; natural terpene blend (D-limonene, |
| | alpha-pinene, beta-pinene, beta- |
| | caryophyllene); carnauba wax |

CBT_{H}

| | |
|---|---|
| Form factor: | Stick - 65 g total mass |
| Flavor: | Menthol/essential oil |
| Active | 1950 mg CBD |
| Ingredient(s): | 10 mg CBC |
| | 40 mg CBG |
| Other | Coconut oil; kester wax; sunflower wax; |
| Ingredient(s): | organic cocoa butter; jojoba oil; rice bran oil; |
| | less than 2% of: essential oil blend; rosemary |
| | extract; menthol |

CBT_{I}

| | |
|---|---|
| Form factor: | Transdermal patch - 2″ × 2″ matrix delivery |
| | system |
| Flavor: | N/A |
| Active | 20 mg CBD, or Full Spectrum Activated CBD |
| Ingredient(s): | and/or other cannabinoid Extract(s) (e.g., |
| | mixtures of CBD & CBN and/or CBD, CBC |
| | and CBG, e.g., in proportions as provided in |
| | other form factors disclosed herein, e.g., in |
| | proportions as provided for herein for other |
| | formulations, such as for tincture or gummy |
| | form discussed herein and/or tabulated herein, |
| | starting from the total cannabinoids being 20 |
| | mg in an embodiment of a transdermal |
| | formulation) |
| | Lidocaine |
| Other | Oleic Acid, Lecithin, Eucalyptus, Natural |
| Ingredient(s): | Terpenes, Propylene Glycol; see also US |
| | Patent Publication No. 20090017120, |
| | incorporated herein by reference, as to |
| | lecithin organogels useful for transdermal |
| | delivery systems (which also include |
| | propylene glycol) |

CBT_{J}

| | |
|---|---|
| Form factor: | Gummy |
| Flavor: | Blueberry |
| Active | 40 mg CBD |
| Ingredient(s): | 5 mg CBC |
| | 5 mg CBG |
| Other | Organic tapioca syrup; organic cane sugar; |
| Ingredient(s): | water; pectin; citric acid; pure tangerine oil; |
| | sodium citrate; color (spirulina, vegetable and |
| | fruit juice); ascorbic acid; carnauba wax |

FIGS. 6 and 7 illustrate example flows of how the hub network 20 may be configured to develop and/or adjust one or more CBTs in accordance with some embodiments. In the example illustrated in FIG. 6, a CBT may be prescribed by the hub network 20 and then modified based on feedback from a subject 32. It should be understood that the subject 32 may be a user of a computing device, such as computing device 100-1, 100-2 illustrated in FIG. 1B. Further, the initial prescription and/or suggestion of a CBT may be provided by the hub network 20 in response to receiving the prescription and/or suggestion from a medical advisor or professional who may enter the prescription or recommendation using one or more computing devices, such as one or more of computing devices 100-3, 100-4, 100-5 of one or more of researcher/advisory/producer network(s) 40 and/or computing devices 100-6, 100-7, 100-8 of provider network(s) 50.

As shown in FIG. 6, an initial CBT, identified as CBT1, may be prescribed or otherwise recommended to subject 32 represented by arrow 602. In some embodiments, the initial CBT1 is provided from hub network 20 and may be based on data collected during a registration and/or enrollment process, such as the enrollment/registration process described above with respect to block 202 in FIG. 2. CBT1 may include one or more active ingredients having a first formulation, such as certain percentage weights and/or ratios of each active ingredient, along with a specific dosing regimen. For example, CBT1 may be prescribed to be used at a certain time (e.g., morning, afternoon, evening) and/or at a certain interval (e.g., a certain time before sleep, periodically throughout the day, etc.). In some embodiments, CBT1 may include one or more of the formulations and/or form factors described above.

The subject 32 may provide feedback to the hub network 20 as indicated by arrow 604. In some embodiments, the feedback 604 may be provided to hub network 20 using an app running on a computing device 100 as described above with respect to block 218 of FIG. 2 and/or FIGS. 4A-4L, for example. As also noted above, the feedback may include or be in the form of sensor data communicated from one or more sensors 70 to hub network 20. For example, subject 32 may provide the feedback 604 to hub network 20 that a single gummy administered before bedtime does not seem to provide the desired effect of aiding in the subject going to sleep.

The feedback 604 received at hub network 20 may be analyzed by hub network 20. In some embodiments, the feedback collected by hub network 20 concerning a subject's experience and/or use of one or more CBTs may analyzed by hub network 20 in combination with data received at hub network 20 from one or more other networks 40, 50 and/or users (medical professionals, researchers, and/or producers). For example, as described above, the hub network 20 may be receive data, including research and/or medical data and/or recommendations from one or more other networks 40, 50. Such data may be analyzed by hub network 20 to suggest a modification to a CBT. The modification may include a modification of one or more active ingredients, inactive ingredients, and/or dosing regimen.

In some embodiments, the hub network 20 may make the recommendation after analyzing the data, but medical approval and/or authorization may be needed before a change in CBT may be communicated to a subject. To facilitate such review and approval, hub network 20 may be configured to cause a request for a change in a CBT, such as for an individual and/or group (e.g., subjects having similar profiles, such as similar gender, age, weight, ailments, prescriptions, medical history, and/or experience with CBTs), to be communicated to an advisory board and/or medical professional, which may be accessible directly on a computing device (e.g., one or more of computing devices 100-3 to 100-8 shown in FIG. 1B) and/or through one or more network(s) 40, 50. As shown in FIG. 6, hub network 20 may transmit an alert, request, and/or other communication requesting a change in a CBT to an advisory board member and/or medical or other prescribing professional, as indicated by arrow 608. Hub network 20 may receive a response to the alert, request, and/or other communication, as indicated by arrow 610 in FIG. 6.

Continuing with the example described above in which a subject provides feedback 604 that a single gummy administered before bedtime is not seeming to provide the desired effect of aiding in the subject going to sleep, a modification to the CBT may be made to try increasing the dosage to two (2) gummies about 30 minutes prior to going to sleep. Assume further that feedback 604 also includes other information such as the subject's age, sex, height, weight, lifestyle information (e.g., coffee drinker, alcoholic beverage consumer, etc.) and other health conditions (in addition to seeking a sleep aid, or comorbidities) and/or such information has already been collected and stored by hub network 20, such as during an enrollment/registration process, and assume further that one of those other health conditions is allergies or a head cold whereby the subject may also be taking a decongestant or a condition where the subject is also taking a stimulant (e.g., coffee drinker or caffeinated beverage consumer), and assume yet further that the subject has increased the dosage to two (2) gummies and yet provides feedback 604 that the single gummy or the two gummies is not effective. Hub network 20 and/or one or more advisory/medical users may use this information in developing a modified CBT, which may result in CBT2 being communicated from hub network 20 to subject 32 (or to a computing device 100 used by subject 32), as indicated by arrow 606 in FIG. 6.

The communication 606 from hub network 20 to subject 32 may include instructions and/or advice that the stimulant or decongestant not be taken within two hours of bedtime (or coffee or caffeinated beverage not to be drank within 4 to 6 hours of going to bed), so that when the gummy or the two gummies are taken at about 30 minutes prior to bedtime, the actives in the gummy/gummies are not minimized by effects of a decongestant or stimulant (or coffee).

Similarly, if the subject feedback 604 is that the gummy is very effective in aiding in sleep, and the additional information of the subject includes that the subject is taking a depressant or drinking alcohol or taking an antihistamine such as diphenhydramine (e.g., Benadryl®), then the communication 606 from hub network 20 to subject 32 may include the instructions and/or advice that alcoholic beverages, or depressants or the antihistamine not be taken within two (2) hours of bedtime so that when the gummy or the two gummies are taken at about 30 minutes prior to bedtime, the actives in the gummy/gummies are not overly maximized and/or that the time for taking the gummy/gummies be adjusted to about an hour before bedtime.

In another example, the feedback 604 may result in a modification and/or adjustment to a formulation of a CBT, e.g., a formulation in which one or more of the active ingredients melatonin, L-theanine, CBD and CBN are increased or decreased by about 5% or 10% by weight (e.g., melatonin increased to about 1.1 mg or decreased to about 0.9 mg and/or L-theanine increased to 55 mg or decreased to 45 mg and/or CBD increased to 44 mg or decreased to 36 mg and/or CBN increased to about 1.1 mg or decreased to about 0.9 mg). Alternatively or additionally, the feedback 604 may result in a modification in a type (e.g., form factor) of a CBT. For example, CBT1 may be gummy and CBT2 may be a liquid or syrup or elixir or tincture formulation (e.g., to have faster action) (or could be a transdermal formulation, such as a transdermal patch or a spot-on formulation to have absorption over a longer period of time).

It should be understood that the feedback 604 received from one or more subjects may be stored and analyzed by hub network 20 and may result in increasing or decreasing dosing of the gummy, adjusting one or more other factors in the subject's life such as when alcohol is consumed or when medicaments are taken, adjusting the gummy formulation to contain more or less of one or more of the actives and/or recommending a different sleep aid formulation (e.g., a formulation containing melatonin, L-theanine, CBD and CBN in a liquid or syrup or elixir or tincture formulation). Thus, the formulation may be adjusted (e.g., adding or subtracting amount w/w of active(s) and/or changing from one form to another, e.g., changing from a solid to a liquid form) and/or adjusting dosing (e.g., adding or subtracting amount of active(s)) and/or adjusting dosing regimen (e.g., time before going to bed-either increasing time or decreasing time and/or amount of gummies taken) and/or adjusting other aspects of the subject's overall health and lifestyle e.g., based on comorbidity(ies) or medications and what is consumed by the subject in addition to the gummy. The dosing regimen could also include where the oral formulation (e.g., gummy and/or liquid/tincture/elixir/syrup) is supplemented with a topical formulation, such as a transdermal patch or a spot-on formulation.

In addition, an advisory/research/medical or other non-subject user can be providing to the hub network 20 information on the benefits of CBD, CBN products based on the subject's age and other information provided by the subject and/or general guidance and information on CBD, CBN products, and that information from the advisory/research/medical or other non-subject user can then be distributed to the subject 32 by the hub network 20. There is thus a flow of information to and from the Advisory/Medical User and to and from the subject pertaining to CBD, CBN products as well as with respect to the particular formulation, dosing and regimen of administration and subject lifestyle and comorbidities and adjustments with respect thereto. It should be understood that the general flow shown in FIG. 6 may be performed iteratively, such that the prescription and/or recommendation of one or more CBTs, feedback, and adjustment of one or more CBTs may be repeated. It should also be understood that the collected data also may be analyzed by one or more provider network(s) 50. For example, one or more provider network(s) 50 may analyze the data to develop new payment and/or service delivery models to cover the cost of one or more CBTs.

In some embodiments, the feedback from one or more subjects may facilitate a change in a CBT (e.g., dosage and/or regimen) for another subject. FIG. 7 provides a high-level flow diagram illustrating such an example in which feedback received from one or more subjects causes a change in a CBT in another subject.

As shown in FIG. 7, an initial CBT, identified as CBT1-1, may be prescribed or otherwise recommended to subject 32-1 represented by arrow 702 and an initial CBT, identified as CBT1-2, may be prescribed or otherwise recommended to subject 32-2 as represented by arrow 708. In some embodiments, the initial CBTs, e.g., CBT1-1, CBT1-2, are provided from hub network 20 and may be based on data collected during a registration and/or enrollment process for each subject 32-1, 32-2, such as the enrollment/registration process described above with respect to block 202 in FIG. 2. CBT1-1 may include one or more active ingredients having a first formulation, such as certain percentage weights and/or ratios of each active ingredients, along with a specific dosing regimen. For example, CBT1-1 may be prescribed to be used at a certain time (e.g., morning, afternoon, evening) and/or at a certain interval (e.g., a certain time before sleep, periodically throughout the day, etc.). CBT1-2 may be the same CBT as CBT1-1 or a different. For example, CBT1-2 may have the same ingredients, dosage (e.g., amount), and/or dosing regimen (e.g., dosing schedule) as CBT1-2, although CBT1-2 may differ from CBT1-1 in one or more of ingredients, dosage, and/or dosing regimen.

One or more subjects 32 (e.g., subjects 32-1, 32-2) may provide feedback to the hub network 20. It should be understood that subjects 32-1, 32-2 are merely exemplary of a plurality, plethora or multitude of subjects (e.g., there could be ten, tens, hundred, hundreds, thousand, thousands, ten thousand, tens of thousands, hundred thousand, hundreds of thousands, etc. of subjects). Subject 32-1 may provide feedback concerning the subject's experience with CBT1-1 to hub network 20, as indicated by arrow 704. In some embodiments, the feedback 704 may be provided to hub network 20 using an app running on a computing device 100 as described above with respect to block 218 of FIG. 2 and/or FIGS. 4A-4L, for example. The feedback also may include or be in the form of sensor data communicated from one or more sensors 70 to hub network 20 as described above. It should be appreciated that other mechanisms of providing feedback, including a telehealth meeting and/or submitting paperwork, may be used.

Additionally or alternatively, subject 32-2 may provide feedback concerning the subject's experience with CBT1-2 to hub network 20, as indicated by arrow 710. The feedback 710 from subject 32-2 to hub network 20 may be provided via an app running on a computing device 100, from one or more sensors 70 associated with subject 32-2, and/or through other suitable means as will be understood by one of ordinary skill in the art. It should be understood that FIG. 7 is merely a high-level schematic and that hub network 20 may communicate with more than two subjects 32.

The hub network 20 may be configured to collect feedback 704, 710 and correlate data to subject populations. For example, patient populations can be based on sex, e.g., male or female, and/or based on subject age; for example, if the subjects are from age 60 to 100, the hub network 20 may be configured to correlate feedback received from the subject population (and information that is provided to the subject population) based on whether each subject is male or female and whether the subject is age 60 to 70 or 60 to 65 and over 65 to 70, and age over 70 to 80 or over 70 to 75 and over 75 to 80, and age over 80 to 90 or over 80 to 85 and over 85 to 90, and over 90 to 100 or over 90 to 95 and over 95 to 100. The correlated information can be further stratified based on subject lifestyle and/or comorbidities, e.g., subjects that drink alcohol, take certain medications (like depressants or medicaments to address anxiety or depression or schizophrenia or high blood pressure or to lower cholesterol such as a statin or to address glucose levels such as a sulfonylurea or insulin) or have certain comorbidities (e.g., have high blood pressure, anxiety, depression, schizophrenia, high cholesterol, diabetes, such as type 1 or type 2). Additional information and/or input may be provided to hub network 20 by a researcher/advisory/medical user, which may include information on gummy dosing or gummy administration (e.g., timing of taking a gummy or other medicaments or consuming alcohol or coffee/caffeine relative to taking a gummy) that is pertinent to particular individuals, e.g., individuals who are male between the ages of 65 and 70 who for their height are normal in weight to slightly overweight (but not more than 30 lbs. overweight or not morbidly obese) and who may have normal to slightly high blood pressure and do not take or may take a blood pressure medicament but who are not diabetic and do not suffer from anxiety or depression or schizophrenia. Thus, the discussion earlier as to FIG. 6 and relative to a single subject can be with regard to a stratified subject population.

The feedback 704, 710 received at hub network 20 may be analyzed by hub network 20. In some embodiments, the feedback collected by hub network 20 concerning the CBT experience reported by subjects 32 may analyzed by hub network 20 in combination with data received at hub network 20 from one or more other networks and/or users. For example, as described above, the hub network 20 may be receive data, including research, clinical, and/or medical data and/or recommendations from one or more other networks 40, 50. Such data may be analyzed by hub network 20 to suggest a modification to a CBT. The modification may include a modification of one or more active ingredients, inactive ingredients, and/or dosing regimen. Additionally or alternatively, the feedback provided by one of the subjects 32 may facilitate a change or modification for another subject 32. In some embodiments, the hub network 20 may be configured to analyze feedback 704, 708 and make one or more recommendations and/or modifications from one or more “if/then” scenarios as to the subjects and subject populations. The following examples of merely illustrative, and it should be understood that hub network 20 may store and/or be configured to apply a plethora of if/then scenarios to collected data.

1. If women respond better with their sleep with when taking the CBD product (e.g., the foregoing exemplary gummy) then for men, a higher dose and/or change in composition of the product may be recommended.

2. If individuals (men and/or women) that weigh less (e.g., are normal or below normal in weight in relation to their height) respond better with their sleep when taking the product (e.g., the foregoing exemplary gummy), then dosing and/or composition may be adjusted based on the subject's or the subject population's weight (e.g., higher dosing for heavier or above normal weight relative to height individuals).

3. If younger (or older) individuals respond better with their sleep when taking the product (e.g., the foregoing exemplary gummy), then the dose and/or composition may be adjusted based on the subject or subject population's age (e.g., increasing dosing as subject or subject population increases in age if younger individuals or populations respond well to a single gummy or decreasing dosing for younger individuals or populations if the older subjects or subject populations respond well to a single gummy).

4. If the sleep variables are no different between the product (e.g., the foregoing exemplary gummy) and melatonin alone (a control) but other factors indicate a beneficial effect (improved cognition, pain relief, improvement in social interactions, etc.), the results then indicate that the cannabinoid (e.g., the foregoing exemplary gummy) is beneficial and different doses of cannabinoid (e.g., adjusting amounts of gummies taken or CBD and CBN amount in a single gummy) and/or adjusting the composition (e.g., adjusting from a gummy to a liquid or elixir or syrup formulation or adjusting from a gummy to a liquid or elixir or syrup formulation and adjusting CBD and/or CBN amount) would be recommended. In addition, in this if/then scenario, there may be a secondary consideration to examine the data to determine if sex, weight, age, etc. also are factors in this analysis, and/or examining the data for coexisting factors (e.g., other health conditions or medicaments taken); and, this/these secondary consideration(s) may obtain a change dosing and/or composition (e.g., adjusting amounts of gummies taken or CBD and CBN amount in a single gummy, and/or adjusting the composition, e.g., adjusting from a gummy to a liquid or elixir or syrup formulation or adjusting from a gummy to a liquid or elixir or syrup formulation and adjusting CBD and/or CBN amount).

5. If the bard data (e.g., RWD and/or RWE) on sleep (e.g., from devices worn by the subject(s) such as FitBits or the like) show that certain stages of sleep are affected differently by the product as compared to melatonin (control), then the hub network 20 (and/or an advisory/medical user) may deduce that the cannabinoids (CDB, CDN) are causing this change and there may then be an examination of the effect as to age, weight, sex, etc. for confounding or influencing factors, leading to dose and/or composition changes (e.g., adjusting amounts of gummies taken or CBD and CBN amount in a single gummy, and/or adjusting the composition, e.g., adjusting from a gummy to a liquid or elixir or syrup formulation or adjusting from a gummy to a liquid or elixir or syrup formulation and adjusting CBD and/or CBN amount).

6. If the hard data on sleep is unchanged but the perceived effect on sleep is improved (Restorative Sleep), then the hub network 20 (and/or an advisory/medical user) may look at the other social, cognitive, etc. data and determine if there is an effect. Depending on the differences with weight, etc. this would influence dosing and/or composition recommendations (e.g., adjusting amounts of gummies taken or CBD and CBN amount in a single gummy, and/or adjusting the composition, e.g., adjusting from a gummy to a liquid or elixir or syrup formulation or adjusting from a gummy to a liquid or elixir or syrup formulation and adjusting CBD and/or CBN amount).

7. The hub network 20 (and/or an advisory/medical user) may be configured to evaluate whether taking the product (e.g., the foregoing exemplary gummy) changes the use of medications, as sleep may affect or may impact many covariant factors (anxiety, cognition, social interaction, pain, etc.). This evaluation may consider any magnitude of the change, and whether the magnitude varies depending on weight, sex, other factors; and, this would influence dosing and/or composition recommendations (e.g., adjusting amounts of gummies taken or CBD and CBN amount in a single gummy, and/or adjusting the composition, e.g., adjusting from a gummy to a liquid or elixir or syrup formulation or adjusting from a gummy to a liquid or elixir or syrup formulation and adjusting CBD and/or CBN amount). Accordingly, it is envisioned that information from subjects, subject populations, and stratified subject populations are used in dosing and/or composition recommendations (e.g., adjusting amounts of gummies taken or CBD and CBN amount in a single gummy, and/or adjusting the composition, e.g., adjusting from a gummy to a liquid or elixir or syrup formulation or adjusting from a gummy to a liquid or elixir or syrup formulation and adjusting CBD and/or CBN amount).

Yet further, positive results from subjects, subject populations, and stratified subject populations, in the form of data that does not violate privacy rights of subjects, subject populations, and stratified subject populations may be provided by the hub network 20 to the medical community and/or governmental regulatory authorities and/or governmental agencies or insurers that are involved in paying for or reimbursing for subject care, e.g., in the form of scientific literature, or reports by advisory/medical user(s) to promote the use of CBTs by subjects, for example as a sleep aid or for covariant factors (anxiety, cognition, social interaction, pain, etc.). Accordingly, it is envisioned that there is promotion of CBTs as a result of the data from subjects, subject populations, and stratified subject populations, as well as information and CBT tailored to a subject as well as to subject population(s) and stratified subject population(s).

As noted above, it should be understood that the data collected and/or analyzed by hub network 20 may be accessed and/or analyzed by one or more other networks, such as a research/advisory/producer network(s) 40 and/or a provider network(s) 50. For example, one or more provider network(s) 50 may analyze the data to develop new payment and/or service delivery models to cover the cost of one or more CBTs.

As shown in FIG. 7, the hub network 20 may suggest another CBT, e.g., CBT1-2, CBT2-2, for each of the subjects 32, as indicated by arrows 706, 712. In some embodiments, CBT1-2 may differ from CBT1-1 based on the feedback received from subject 32. For example, if feedback 704 from the subject 32-1 indicates that the CBT1-1 is too effective (e.g., the subject feels drowsy, cannot stay awake, and/or has difficulty focusing) or is ineffective (e.g., the subject cannot sleep and/or relax), then the CBT for subject 32-1 may be modified based on the feedback 704 from the subject 32-1. In a similar manner, if feedback 710 from subject 32-2 indicates that the CBT2-1 is too effective and/or ineffective, then the CBT for subject 32-2 may be modified based on the feedback 710 from subject 32-2.

As noted above, the feedback from one subject (e.g., subject 32-1) may precipitate a change in the CBT for another subject (e.g., subject 32-2). For example, it should be understood that the feedback from subjects may be received at hub network 20 at various times, as the feedback may be provided in real-time to the hub network 20 (e.g., the feedback may be transmitted as the subject enters the feedback in the app running on a computing device 100). Thus, if different subjects enter feedback at different times, then the feedback may be received at the hub network 20 at different times. However, it should be understood that feedback may be received at different times even if the hub network 20 batch collects feedback from enrolled and/or registered subjects, although some feedback may be received simultaneously by hub network 20.

Referring to the example illustrated in FIG. 7, if the feedback 710 from subject 32-2 is received before the feedback 704 from subject 32-1, then the hub network 20 may analyze the feedback 710 and determine that a CBT should be adjusted for subject 32-2, as well as other subjects that may have similar profiles (e.g., similar gender, age, weight, ailments, medical history, prescriptions, and/or experience with CBTs, etc.). Hub network 20 may then recommend a different CBT for subject 32-2, e.g., CBT2-2, at some time after receiving feedback 710. In some embodiments, the hub network 20 may make the recommendation after analyzing the data, but medical approval and/or authorization may be needed before a change in CBT may be communicated to a subject. To facilitate such review and approval, hub network 20 may be configured to cause a request for a change in a CBT, such as for an individual and/or group (e.g., subjects having similar profiles, such as similar gender, age, weight, ailments, prescriptions, medical history, and/or experience with CBTs), to be communicated to an advisory board and/or medical professional.

As shown in FIG. 7, hub network 20 may transmit an alert, request, and/or other communication requesting a change in a CBT to an advisory board member and/or medical or other prescribing professional, as indicated by arrow 708. Hub network 20 may receive a response to the alert, request, and/or other communication, as indicated by arrow 716 in FIG. 7. If the change in one or more CBTs is approved, then the hub network 20 may communicate transmit a message to subject 32-2 with a new CBT, e.g., CBT2-2, as indicated by arrow 712. In some embodiments, the change may not be approved and/or hub network 20 and thus CBT2-2 may be the same as CBT2-1.

As noted above, in some embodiments, the feedback 704 from subject 32-1 may be received after the feedback 710 and/or the approval of a change in CBT from an advisory board member and/or medical or prescribing professional shown by arrow 716. Consequently, in response to feedback 704, hub network 20 may transmit an instruction to modify and/or change a CBT automatically to subject 32-1 upon receiving analyzing feedback 704. For example, hub network 20 may receive and analyze feedback 704 and determine that subject 32-1 has a similar (or the same) profile, and thus the same changes in one or more CBTs made for subject 32-2 should be made to the one or more CBTs for subject 32-1. Accordingly, CBT2-2 may be the same as CBT1-2, although CBT1-2 and CBT2-2 may be different as will be understood by one of ordinary skill in the art. It also should be understood that the general flow shown in FIG. 7 may be performed iteratively, such that the prescription and/or recommendation of one or more CBTs, feedback, and adjustment of one or more CBTs may be repeated.

As noted above, various types of adjustments may be made to a CBT, including changes to a dosage regimen, ingredients, and formula. A formula may be adjusted by increasing and/or decreasing one or more active and/or inactive ingredients of a CBT. The adjustment may be made on a percentage weight basis, as a ratio of one active/inactive ingredient to another active/inactive ingredient, or otherwise as will be understood by one of ordinary skill in the art. Additionally or alternatively, a dosing regimen may be adjusted. The dosing regimen may adjust a time of day and/or frequency for a CBT. In some embodiments, the dosing regimen may be adjusted to avoid a time at which a subject takes other medications or to coincide with the consumption of other medications. Other modifications to the CBT will be understood by one of ordinary skill in the art.

For example, hub network 20 may determine, based on analysis, that if women respond better with their sleep when taking a CBD-based product than men, then the hub network 20 may suggest a higher dosage (e.g., two gummies instead of one) and/or a change in composition of the product for men. In another example, if hub network 20 determines, based on analysis of data stored by hub network 20, a subject of a certain gender having a height and weight in a specific range responds better (e.g., has better sleep, less pain, more focus, etc.) than individuals of the same gender of the same height but in another weight range, then a change in dosage and/or change in composition may be recommended. In yet another example, if analysis of data by hub network 20 identifies a correlation between age and effectiveness and/or safety (e.g., younger individuals respond better to higher doses and older individuals respond better to lower doses), then hub network. 20 may recommend a change to either a dosage and/or a composition of CBT.

As referenced above, the hub network 20 may receive sensor data, such as data from one or more of a fitness tracker, mattress/bed sensor, and/or medical device, and may determine that certain stages of sleep are affected differently by different compositions of a CBT based on the received sensor data. The hub network 20 may be configured to identify such trends in the data and generate one or more alerts and/or recommendations that may be transmitted to one or more researchers and/or medical or prescribing personnel. Advantageously, because hub network 20 is configured to receive and analyze real-world data as well as data from researchers, medical personnel, and/or cannabinoid producers, the hub network 20 may be configured to identify the effects based on age, weight, sex, and/or other influencing factors on the safety and/or efficacy of one or more CBTs.

Additionally and/or alternatively, hub network 20 may also be configured to identify trends and/or relationships between the safety and efficacy of one or more CBTs and a product and/or producer. For example, a producer and/or manufacturer of a cannabinoid-based treatment or product may provide data concerning, growing conditions, cannabis strains, and/or crop planting and/or harvesting to hub network 20, which data may be traceable to a particular CBT provided to a particular subject and/or group of subjects. Consequently, hub network 20 may be configured to analyze this data in addition to the real-world data received from the one or more subjects 32 to determine efficacy and/or safety of a CBT.

The present disclosure can be embodied in the form of methods and apparatuses for practicing those methods. The present disclosure can also be embodied in the form of program code embodied in tangible media, such as secure digital (“SD”) cards, USB flash drives, diskettes, CD-ROMs, DVD-ROMs, Blu-ray disks, hard drives, or any other non-transitory machine-readable storage medium, wherein, when the program code is loaded into and executed by a machine, such as a computer or processor, the machine or processor becomes an apparatus for practicing the disclosure. The present disclosure can also be embodied in the form of program code, for example, whether stored in a storage medium, loaded into and/or executed by a machine, or transmitted over some transmission medium, such as over electrical wiring or cabling, through fiber optics, or via electromagnetic radiation, wherein, when the program code is loaded into and executed by a machine, such as a computer or processor, the machine becomes an apparatus for practicing the disclosure. When implemented on a general-purpose processor, the program code segments combine with the processor to provide a unique device that operates analogously to specific logic circuits.

Furthermore, the present disclosure, especially with respect to the herein discussion as to FIGS. 6 and 7, envisions methods of administration and use of formulations for use in methods of administration, said methods including as described in those paragraphs, including where the subject is administering liquid (e.g., tincture, elixir or syrup) or solid (e.g. gummy) or transdermal (e.g., patch or spot-on) forms himself or herself, or they are administered to or into the subject, and there is then variance of further administration based on the subject or the subject's group providing feedback to and receiving information from the Advisory/Medical User; and including to address issues of sleep, e.g., to induce sleep, as well as for other conditions mentioned herein, such as anxiety, depression, joint pain, inflammation. Thus, in the above discussion, especially as to FIGS. 6 and 7 the condition for which the subject is self-administering or having administration of the CBT can be, instead of issues of sleep, another herein condition such as anxiety, depression, joint pain, inflammation. Furthermore, while the above discussion, especially as to FIGS. 6 and 7, focus on initial dosing being a gummy, the initial dosing can be a liquid (e.g., tincture, elixir or syrup) or a transdermal (e.g., patch or spot-on). In such instances, the switching or augmenting of a formulation can be then to another herein-disclosed formulation, such as gummy. Moreover, a thrust of this disclosure is a method of administration of CBT formulation(s) or use of CBT formulation(s) in a method of administration in accordance with the herein discussion of FIGS. 6 and 7, e.g., wherein there is an initial provision of a first CBT formulation and information to a subject (or a subject population) and the subject (or individuals of the subject population) either self-administers or has administration according to an initial regimen, and there is feedback by the subject (or a population including the subject) and feedback to the subject (or a population including the subject) from the Advisory/Medical User whereby there is another provision of a second CBT formulation and information, whereby the second CBT formulation and information can be the same as or analogous to the first CBT formulation and information, or dosing and/or formulation etc. (e.g., as discussed above as to FIGS. 6 and 7) is adjusted based on the feedback, with the subject (or subject population) self-administering or having administration of the second CBT formulation; and, this process can continue to third CBT formulation and information, fourth CBT formulation and information, and onward to further formulations and information until the subject's situation or condition (or the subject population's situation or condition) is adequately addressed—with it understood that CBT formulations and information regarding same may also vary over time as the subject increases in age and the subject's other conditions (or comorbidities) change (e.g., when the subject shifts from one subject population into another subject population based on age and/or other factors).

The above-described embodiments are merely possible examples of implementations, and merely set forth a clear understanding of the principles of the disclosure. Many variations and modifications may be made to the above-described embodiments and examples of the disclosure without departing substantially from the spirit and principles of the disclosure. All such modifications and variations are intended to be included herein within the scope of this disclosure and the present disclosure and protected by the following claims.

While this specification contains many specifics, these should not be construed as limitations on the scope of any disclosure or of what may be claimed, but rather as descriptions of features that may be specific to particular embodiments or examples of particular disclosures. Certain features that are described in this specification in the context of separate embodiments or examples may also be implemented in combination in a single embodiment or example. Conversely, various features that are described in the context of a single embodiment may also be implemented in multiple embodiments or examples separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

While various embodiments have been described, it is to be understood that the embodiments described are illustrative only and that the scope of the subject matter is to be accorded a full range of equivalents, many variations and modifications naturally occurring to those of skill in the art from a perusal hereof.

1. A system, comprising:
a non-transitory machine-readable storage medium; and
a processor in communication with the non-transitory machine-readable storage medium, the processor configured to:
receive first data concerning a first subject's experience with a first cannabinoid-based treatment;
store the first data to be stored in the non-transitory machine-readable storage medium in a profile associated with the first subject;
identify at least one change to be made to the first cannabinoid-based treatment; and
cause at least one message identifying a second cannabinoid-based treatment to be generated in response to identifying the at least one change to be made to the first cannabinoid-based treatment.

2. The system of claim 1, wherein the processor is configured to identify the second cannabinoid-based treatment in response to:
analyzing the first data; and
analyzing second data concerning a second subject's experience with the first cannabinoid-based treatment.

3. The system of claim 2, wherein the second cannabinoid-based treatment differs from the first cannabinoid-based treatment by at least one of:
an amount of at least one active ingredient; or
a dosing regimen.

4. (canceled)

5. (canceled)

6. The system of claim 2, wherein the second cannabinoid-based treatment differs from the first cannabinoid-based treatment by at least one of:
an amount of at least one active ingredient; or
a dosing regimen.

7. (canceled)

8. The system of claim 2, wherein the second data is stored in the non-transitory machine-readable storage medium in a profile associated with the second subject.

9. The system of claim 1 wherein;
the first data is received from a computing device associated with the first subject; and
the at least one message is transmitted to the computing device associated with the first subject.

10. (canceled)

11. The system of claim 1, wherein the first data is received from a first sensor associated with the first subject.

12. The system of claim 1, wherein the at least one message is transmitted to at least one of a medical professional and a member of an advisory board.

13. The system of claim 1, wherein the first subject is at least 65 years old.

14. The system of claim 1, wherein the processor is configured to;
receive real-world data concerning a plurality of cannabinoid-based treatments from a plurality of subjects; and
create a profile for each subject of the plurality of subjects in the non-transitory machine-readable storage medium.

15. (canceled)

16. The system of claim 14, wherein each subject of the plurality of subjects is at least 65 years of age.

17. A system, comprising:
a first computing device including an input device, a display, a communication interface, and a first processor in communication with the input device, the display, and the communication interface, the first processor configured to:
receive, from the input device, first data concerning a first subject's experience with a first cannabinoid-based treatment;
cause the first data to be transmitted, using the communication interface, to a second computing device;
receive, using the communication interface, a message identifying a second cannabinoid-based treatment; and
cause a notification identifying the second cannabinoid-based treatment to be displayed on the display.

18. The system of claim 17, wherein the second cannabinoid-based treatment differs from the first cannabinoid-based treatment by an amount of at least one active ingredient and by a dosing regimen.

19. (canceled)

20. The system of claim 17, wherein;
the first computing device is configured to receive health data from a sensor associated with the first subject, and
the health data includes at least one of a pulse rate, a blood pressure, a blood-oxygen level, and data about the first subject's sleep.

21. (canceled)

22. (canceled)

23. A treatment method, comprising:
administering a first cannabinoid-based treatment to a first subject;
receiving feedback from the first subject after the first cannabinoid-based treatment has been administered;
adjusting the first cannabinoid-based treatment to provide a second cannabinoid-based treatment in response to the feedback; and
administering the second cannabinoid-based treatment to the first subject.

24. The treatment method of claim 23, wherein;
the first subject is at least 65 years of age; and
the feedback concerns an ailment of aging.

25. (canceled)

26. The treatment method of claim 23, wherein the second cannabinoid-based treatment differs from the first cannabinoid-based treatment by an amount of at least one active ingredient on a percentage weight basis.

27. (canceled)

28. (canceled)

29. The treatment method of claim 23, wherein the second cannabinoid-based treatment differs from the first cannabinoid-based treatment by a dosing regimen.

30. The treatment method of claim 29, wherein a frequency of the first cannabinoid-based treatment is different from a frequency of the second cannabinoid-based treatment.

31. (canceled)

32. The treatment method of claim 29, wherein a time of day for the first cannabinoid-based treatment is different from a time of day for the second cannabinoid-based treatment.

33. (canceled)
